(19) United States
(12) Patent Application Publication
(10) Pub. No.: US 2008/0234498 A1
(43) Pub. Date: Sep. 25, 2008

(54) Pyrovalerone Analogues and Therapeutic Uses Thereof

(75) Inventors: David Butler, Medford, MA (US); Bertha K. Madras, Newton, MA (US); Peter C. Meltzer, Lexington, MA (US)

Correspondence Address: EDWARDS ANGELL PALMER & DODGE LLP, P.O. BOX 55874, BOSTON, MA, 02205, US

(73) Assignees: PRESIDENT AND FELLOW OF HARVARD COLLEGE, Cambridge, MA (US); ORGANIX, INC., Woburn, MA (US)

(21) Appl. No.: 10/575,177

(22) PCT Filed: Oct. 8, 2004
(86) PCT No.: PCT/US04/33349

§ 371 Date: Sep. 11, 2007

Related U.S. Application Data
(60) Provisional application No. 60/509,882, filed on Oct. 8, 2003.

(51) Int. Cl. C07D 207/00

(52) U.S. Cl. 548/570

(57) ABSTRACT

New compounds that bind to monoamine transporters are described. The compounds of the present invention can be racemic or pure R— or S-enantiomers. Certain preferred compounds of the present invention have a high selectively dopamine transporter versus the serotonin transporter. Preferred monoamine transporters for the practice of the present invention include the dopamine transporter, the serotonin transporter and the norepinephrine transporter.

### RELATED APPLICATIONS

This application claims benefit of priority to U.S. Provisional Patent Application No. 60/509,882, filed Oct. 8, 2003, the contents of which are hereby incorporated by reference in their entirety.

### GOVERNMENT SUPPORT

This invention was made with support from NIH grant Nos. DA00304, DA06303, DA11558, DA1530, DA18825, and NO1 DA1-8825. The U.S. Government may have certain rights in this invention.

### FIELD OF THE INVENTION

The present invention relates to novel compounds that have an affinity for a monoamine transporter, e.g., the dopamine transporter (DAT), or norepinephrine transporter (NET). Such agents can be useful for the early diagnosis and treatment of diverse neurological and psychiatric conditions.

### BACKGROUND OF THE INVENTION

Monoamine transporters play a variety of roles, and compounds with affinity for the monoamine transporters have been proposed for therapy and/or diagnosis of medical indications that include (but are not limited to) attention deficit hyperactivity disorder (ADHD), Parkinson's disease, cocaine addiction, smoking cessation, weight reduction, obsessive-compulsive disorder, various forms of depression, traumatic brain injury, stroke, and narcolepsy. Examples of monoamine transporters include, e.g., the dopamine transporter (DAT), serotonin transporter (SERT) or norepinephrine transporter (NET).

Therapies for treating diseases and disorders related to monoamine transport are needed. For example, there is a need for protective agents for neurodegenerative diseases such as Parkinson's disease and Alzheimer's disease as well as therapeutic agents for dopamine related dysfunction such as Attention Deficit Disorder. Compounds that inhibit monoamine reuptake in the mammalian system are sought to provide such therapies.

Other neuropsychiatric disorders, including Tourette's Syndrome and Lesch Nyhan Syndrome and possibly Rett's syndrome, are also marked by changes in DAT density. The DAT also is the target of the most widely used drug for attention deficit disorder, methylphenidate. The capacity to monitor the transporter in persons suffering from this disorder can have diagnostic and therapeutic implications.

The density of the DAT in the brains of substance abusers has also been shown to deviate from that in normal brain. For example, the density is elevated in post-mortem tissues of cocaine abusers (Little et al., Brain Res. 1993, 628, 17-25). On the other hand, the density of the DAT in chronic nonviolent alcohol abusers is decreased markedly. (Tiihonen et al., Nature Medicine 1995, 1, 654-657). Brain imaging of substance abusers can be useful for understanding the pathological processes of cocaine and alcohol abuse and monitoring restoration of normal brain function during treatment.

Accordingly, compounds that bind to the DAT provide important clinical information to assist in the diagnosis and treatment of these and other DAT related disease states. Serotonin (5-hydroxytryptamine) neurotransmission is regulated and terminated by active transport via the serotonin transporter (SERT). Inhibition of 5-hydroxytryptamine reuptake has an effect on diseases mediated by 5HT receptors. Compounds that provide such inhibition can be useful, for example, as therapeutic anti-depressants. Structurally related to dopamine and norepinephrine transporters (Nelson N. 1998. J. Neurochem 71:1785-1803), the SERT is the primary site of action of diverse antidepressant drugs, ranging from tricyclics such as imipramine and amitriptyline, to serotonin selective reuptake inhibitors (SSRI's) such as citalopram, fluoxetine and sertraline.

Antidepressant drugs delay the removal of extracellular serotonin from the synapse by blocking serotonin transport, thereby prolonging the duration of serotonin receptor activity. The increased availability of serotonin triggers a cascade of neuroadaptive processes, which produces symptom relief after two to four weeks. Presently known antidepressants also produce certain side effects and may selectively alleviate specific symptoms of depression (Nestler E J. 1998. Biol Psychiatry 44:526-533). Thus, it is desirable to develop novel antidepressants. The majority of clinically approved drugs to treat depression or obsessive-compulsive disorder are high affinity inhibitors of serotonin and/or norepinephrine transport.

Norepinephrine regulates mood, is involved in leaning and memory, and controls endocrine and autonomic functions. Dysfunction of norepinephrine neurotransmission has been implicated in depression, cardiovascular and thermal pathophysiology. The norepinephrine transporter (NET) regulates extracellular levels of norepinephrine in brain, in heart, and in the sympathetic nervous system. Clinically, the norepinephrine transporter is a principal target of selective or non-selective anti-depressant drugs and stimulant drugs of abuse such as cocaine and amphetamines. Blockade of the norepinephrine transporter is implicated in appetite suppression. Gehlert et al. J. Pharmacol. Exp. Ther. 287:122-127 (1998). Imaging of the norepinephrine transporter may also be useful for viewing the status of sympathetic innervation in the heart and in other adrenergic terminals, and for detecting neuroblastomas. Hadrich et al. J. Med. Chem. 42:3010-3018 (1999); Raffel et al., J. Nucl. Med. 40:323-330 (1999).

Monoamine transporters such as, the dopamine transporter, serotonin transporter and norepinephrine transporter, are localized on monoamine nerve terminals. Compounds that bind to these sites can be useful as (i) probes for neuro-degenerative diseases (e.g., Parkinson's disease), (ii) therapeutic drugs for neurodegenerative diseases (e.g., Parkinson's and Alzheimer's disease), (iii) therapeutic drugs for dopamine dysfunction (e.g., Attention Deficit Disorder), (iv) treatment of psychiatric dysfunction (e.g., depression) and (v) treatment of clinical dysfunction (e.g., migraine).

It is desirable to avoid unwanted side effects of treatments targeting monoamine transporters, to the extent possible. It is also desirable to produce efficient and effective diagnostics for various conditions involving monoamine transporters.

Furthermore, it would be useful to improve the bioavailability of compounds used to treat and/or diagnose monoamine transporter related diseases and disorders. It would be useful to modify these compounds to block or reduce metabolism of the compounds, while maintaining, or ideally, improving potency and/or selectivity of the compounds.

### SUMMARY OF THE INVENTION

The present invention relates to compounds that bind and/or inhibit monoamine transporters such as the dopamine, serotonin and norepinephrine transporters of mammalian systems.

More specifically, the invention relates to compounds, such as pyrovalerone analogs, that are active (as racemates or purified enantiomers) in monoamine uptake systems and are selective for different monoamine uptake systems such as DAT, NET, and SERT. For example, an enantiomer, 2S-pyrovalerone (see Scheme I) is potent at DAT, (IC₅₀=3 nM) and selective at SERT (IC₅₀>4 μM).

Compounds of the invention are represented by the following general formulae:

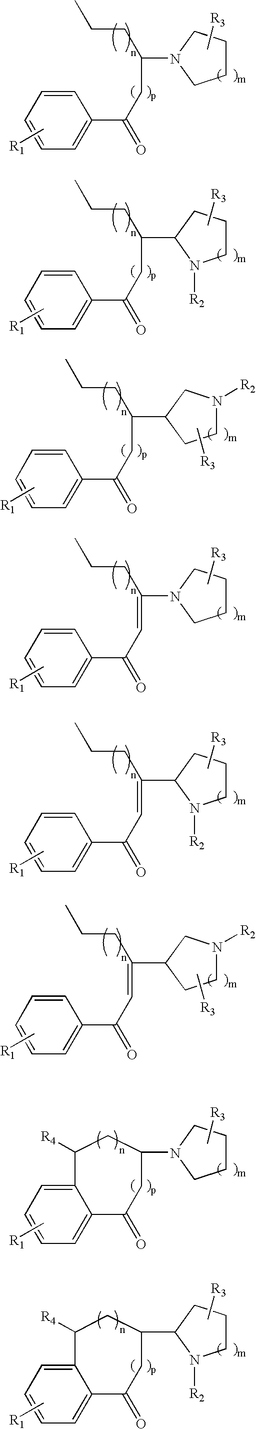

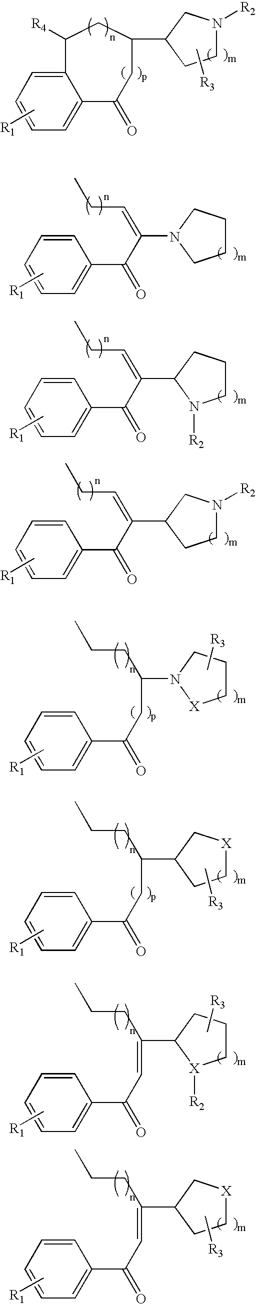

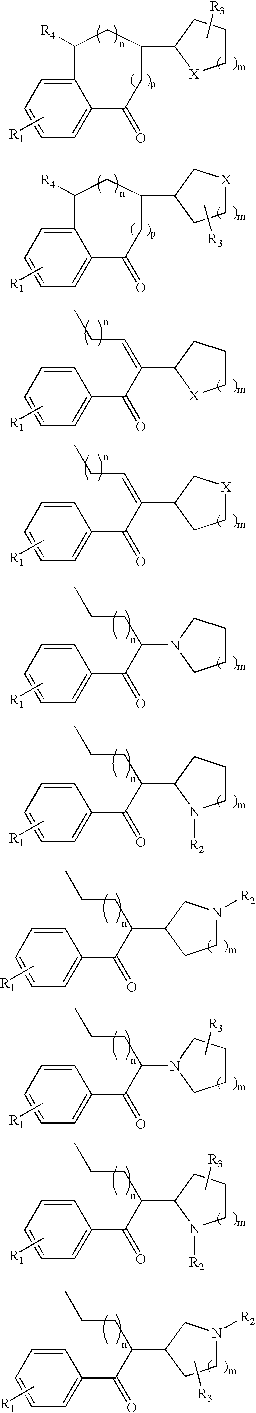

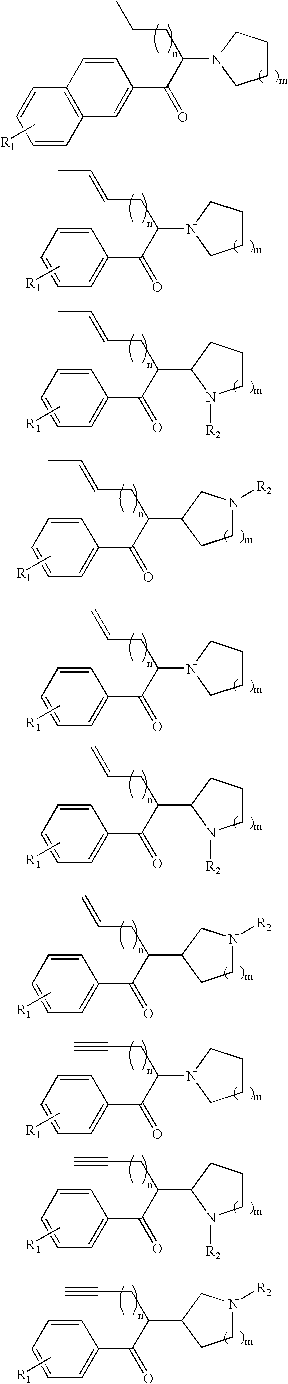

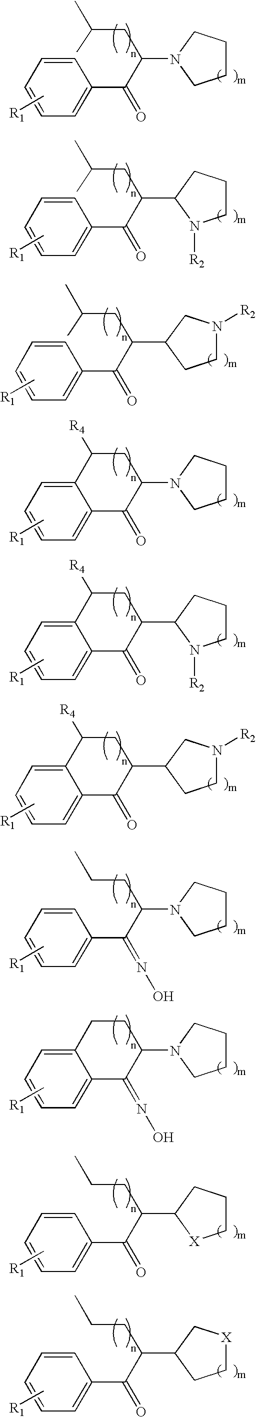

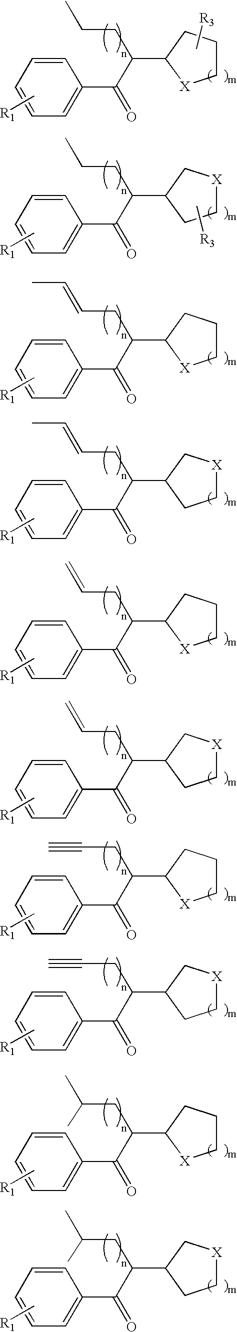

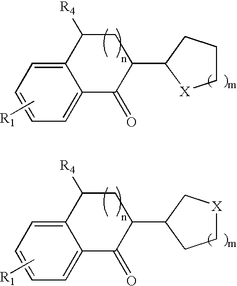

wherein,
R₁=one to four substituents independently selected from the group consisting of H, halogen (preferably F, Br, Cl, or I), substituted or unsubstituted alkyl (preferably methyl, ethyl, isopropropyl, isobutyl, or t-butyl), substituted or unsubstituted alkoxy (preferably methoxy), substituted or unsubstituted alkenyl, substituted or unsubstituted alkenyloxy, substituted or unsubstituted alkynyl, substituted or unsubstituted alkynyloxy, (CH₂)ₙ—Ar, OH, OC(O)-alkyl (preferably methyl); CF₃; NO₂; NH₂; CN; NHCOCH₃; CO-alkyl (more preferably COCH₃), CH₂OH, (CH₂)ₙOR₂ (in which n is 1 to 4) and (CH₂)ₙOCOR₂; (in which n is 1 to 4);
R₂═H, substituted or unsubstituted alkyl (preferably methyl, ethyl, isopropropyl, isobutyl, or t-butyl), substituted or unsubstituted alkoxy (preferably methoxy), substituted or unsubstituted alkenyl, substituted or unsubstituted alkenyloxy, substituted or unsubstituted alkynyl, substituted or unsubstituted alkynyloxy, or CH₂ArR₁;
R₃=one or two substituents independently selected from the group consisting of H, halogen (preferably F, Br, Cl, or I), substituted or unsubstituted alkyl (preferably methyl, ethyl, isopropropyl, isobutyl, or t-butyl), substituted or unsubstituted alkoxy (preferably methoxy), substituted or unsubstituted alkenyl, substituted or unsubstituted alkenyloxy, substituted or unsubstituted alkynyl, substituted or unsubstituted alkynyloxy, OH, (CH₂)ₙArR₁; CF₃; NO₂; NH₂; CN; NHCOCH₃, CO-alkyl (preferably COCH₃), CH₂OH, (CH₂)ₙOR₂ (in which n is 1 to 4) and (CH₂)ₙOCOR₂; (in which n is 1 to 4);
R₄═H, halogen (preferably F, Br, Cl, or I), substituted or unsubstituted alkyl (preferably methyl), substituted or unsubstituted alkoxy (preferably methoxy), substituted or unsubstituted alkenyl, substituted or unsubstituted alkenyloxy, substituted or unsubstituted alkynyl, substituted or unsubstituted alkynyloxy, OH, OC(O)-alkyl (preferably methyl); CF₃; NO₂; NH₂; CN; NHCO-alkyl (preferably NHCOCH₃), COCH₃, CH₂OH, (CH₂)ₙOR₂ (in which n is 1 to 4) and (CH₂)ₙOCOR₂; (in which n is 1 to 4);
Ar is an aromatic group (preferably phenyl or naphthyl);
n=0-4;
m, p=0-2; and
X═O, CH₂, S, SO₂, or SO; or a pharmaceutically acceptable salt of the compound;
   with the proviso that, when the compound is a racemic mixture, the compound is not α-pyrrolidino-valerophenone, 1-(p-methyl-phenyl)-2-pyrrolidino-pentan-1-one (also known as pyrovalerone), 1-phenyl-2-pyrrolidino-3-methylbutan-1-one, 1-(p-methoxy-phenyl)-2-pyrrolidino-pentan-1-one, 1-(p-hydroxy-phenyl)-2-pyrrolidino-pentan-1-one, 1-phenyl-2-pyrrolidino-butan-1-one, 1-phenyl-2-pyrrolidino-heptan-1-one, 1-(p-chloro-phenyl)-2-pyrrolidino-pentan-1-one, 1-(m-methyl-phenyl)-2-pyrrolidino-pentan-1-one, 1-phenyl-2-pyrrolidino-nonan-1-one, 1-(p-methoxy-phenyl)-2-pyrrolidino-hexan-1-one, or α-(2′-methyl-pyrrolidino)-valerophenone.

In preferred embodiments, R₁ represents F (at the 2, 3 or 4 position); Cl (at the 2, 3 or 4 position); I (at the 2, 3 or 4 position) 3,4-diCl; 3-C1,4-C(CH₂)CH₃; 3-Br,4-isopropyl; 3-I,4-C(CH₂)CH₃; 4-C1,3-C(CH₂)CH₃; 4-Br,3-isopropyl; 4-I,3-isopropyl; 3,4-diOH; 3,4-diOAc; 3,4-diOCH₃; 3-OH,4-Cl; 3-OH,4-F; 3-OAc,4-Cl; 3-OAc,4-F; 3-C1,4-OH; 3-F,4-OH; 3-C1,4-OAc; or 3-F,4-OAc. In certain preferred embodiments, R₁ is an aromatic group.

The invention also provides additional compounds, including compounds represented by Formulas I and U, as described hereinbelow.

The compounds of the present invention can be racemic or pure R— or S-enantiomers. Thus, the structural formulae illustrated herein are intended to represent each enantiomer and diastereomer of the illustrated compound, and mixtures thereof, unless stated otherwise. The invention also includes salts, hydrates, and tautomeric forms of the compounds of the invention unless stated otherwise.

The compounds of the present invention can be radiolabeled, for example, to assay cocaine receptors. Certain preferred compounds of the present invention have a high selectivity for the DAT versus the SERT. Preferred compounds have an IC₅₀ SERT/DAT ratio of greater than about 10, preferably greater than about 30 and more preferably 50 or more. In addition, preferably the compounds have an IC₅₀ at the DAT of less than about 500 nM, preferably less than 60 nM, more preferably less than about 20 nM and most preferably less than about 3 nM.

The present invention also provides pharmaceutical therapeutic compositions comprising the compounds formulated in a pharmaceutically acceptable carrier.

Preferred monoamine transporters for the practice of the present invention include the dopamine transporter, the serotonin transporter and the norepinephrine transporter.

In a preferred embodiment, the invention also provides a method for inhibiting dopamine reuptake of a dopamine transporter by contacting the dopamine transporter with a dopamine reuptake inhibiting amount of a compound of the present invention. Inhibition of dopamine reuptake of a dopamine transporter in a mammal is provided in accord with the present invention by administering to the mammal a dopamine inhibiting amount of a compound of the present invention in a pharmaceutically acceptable carrier. FIG. 1 is illustrative of the compounds of the present invention such as analogs of pyrovalerone, that have activity in monoamine uptake systems and are selective for different monoamine uptake systems such as DAT, NET, and SERT. For example, an enantiomer, 2S-pyrovalerone (see Scheme I) is potent at DAT, (IC₅₀=3 nM) and selective at SERT (IC₅₀>4 μM).

The invention also relates to a method for treating a mammal having a disorder selected from neurodegenerative disease, psychiatric dysfunction, dopamine dysfunction, cocaine abuse and clinical dysfunction comprising administering to the mammal an effective amount of a compound of the present invention. In certain methods, the neurodegenerative disease is selected from Parkinson's disease and Alzheimer's disease. An example of a psychiatric disorder which can be treated by the present methods is depression.

The invention also relates to methods for treating dopamine related dysfunction in a mammal comprising administering to the mammal a dopamine reuptake inhibiting amount of a compound as described herein. An example of a dopamine related dysfunction is Attention deficit disorder.

The invention also relates to methods for treating serotonin related dysfunction in a mammal comprising administering to the mammal a serotonin reuptake inhibiting amount of a compound as described herein.

The invention also relates to methods for treating norepinephrine related dysfunction in a mammal comprising administering to the mammal a norepinephrine reuptake inhibiting amount of a compound as described herein.

In the above described methods, when reference is made to a compound of the invention, it will be understood that combinations of two or more compounds of the invention may also be used.

The term “lower alkyl” when used herein designates saturated branched or straight chain hydrocarbon monovalent substituents containing from 1 to about 8 carbon atoms such as methyl, ethyl, isopropyl, n-propyl, n-butyl, (CH₂)ₙCH3, C(CH₃)₃; etc., more preferably 1 to 4 carbons. The term “lower alkoxy” designates lower alkoxy substituents containing from 1 to about 8 carbon atoms such as methoxy, ethoxy, isopropoxy, etc., more preferably 1 to 4 carbon atoms.

The term “lower alkenyl” when used herein designates aliphatic unsaturated branched or straight chain vinyl hydrocarbon substituents containing from 2 to about 8 carbon atoms such as allyl, etc., more preferably 2 to 4 carbons. The term “lower alkynyl” designates lower alkynyl substituents containing from 2 to about 8 carbon atoms, more preferably 2 to 4 carbon atoms such as, for example, propyne, butyne, etc.

The term “aliphatic” is art-recognized and as used herein includes alkyl, alkenyl, and alkynyl groups as described above.

The terms “substituted lower alkyl,” “substituted lower alkoxy,” “substituted lower alkenyl” and “substituted lower alkynyl,” when used herein, include corresponding alkyl, alkoxy, alkenyl or alkynyl groups substituted with halide, hydroxy, carboxylic acid, or carboxamide groups, etc. such as, for example, —CH₂OH, —CH₂CH₂COOH, —CH₂CONH₂, —OCH₂CH₂OH, —OCH₂COOH, —OCH₂CH₂CONH₂, etc. As used herein, the terms lower alkyl, lower alkoxy, lower alkenyl and lower alkynyl are meant to include where practical substituted such groups as described above.

The term “aromatic” (or “aryl”) is art-recognized, and as used herein, refers to a carbocyclic or heterocyclic aromatic ring moiety. Aromatic ring systems include polycyclic aromatic systems such as naphthyl, benzofuranyl, and the like. Preferred aromatic moieties have 5 to 10 atoms in the aromatic ring system and may include 0 to 4 heteroatoms selected from the group consisting of O, N, and S. Examples of aromatic moieties include phenyl, naphthyl, furanyl, pyrrolyl, thiophenyl, indolyl, pyridyl, pyrazolyl, pyrazinyl, benzofuranyl, tetrazolyl, isoxazolyl, and the like. Aromatic groups may be unsubstituted or substituted with 1 to 4 substituents, including alkyl, halogen, hydroxyl, and the like.

The term “substantially enantiomerically pure”, as used herein in reference to an enantiomer of a compound, refers to an enantiomer (e.g., the (S)-enantiomer) which is substantially free of the corresponding enantiomer (e.g., the (R)-enantiomer), i.e., not a racemic mixture of enantiomers. In preferred embodiments, an enantiomer which is substantially enantiomerically pure is present is greater than about 80% enantiomeric excess (e.e.), more preferably greater than about 90%, 95%, or 98% e.e.

When X (a ring substituent in certain of the formulae above) contains a carbon atom as the ring member, reference to X is sometimes made herein as a carbon group. Thus, when X is a carbon group, as that phrase is used herein, it means that a carbon atom is a ring member at the X position.

### BRIEF DESCRIPTION OF THE FIGURES

FIG. 1 is a chart showing the compounds of the invention and their Kᵢ with respect to DAT, SERT and NET.

### DETAILED DESCRIPTION OF THE INVENTION

In accord with the present invention, novel tropane compounds are provided that bind to monoamine transporters, preferably the DAT. Certain preferred compounds also have a high selectivity for the DAT versus the SERT. Preferred compounds of the invention include those having the formula:

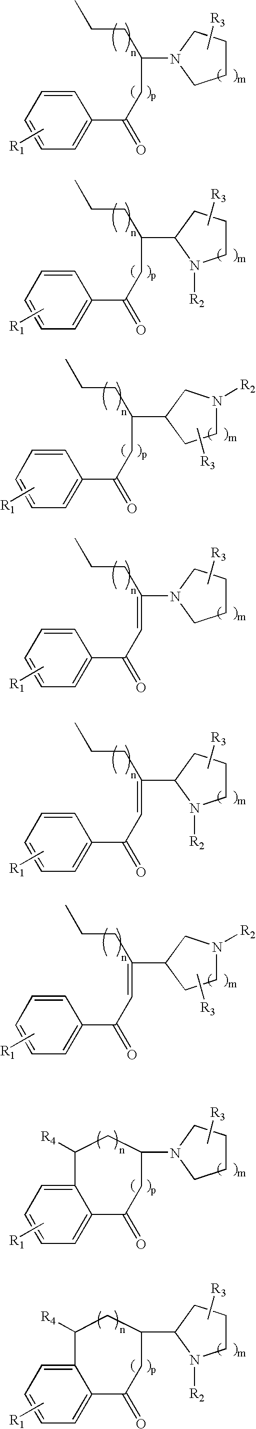

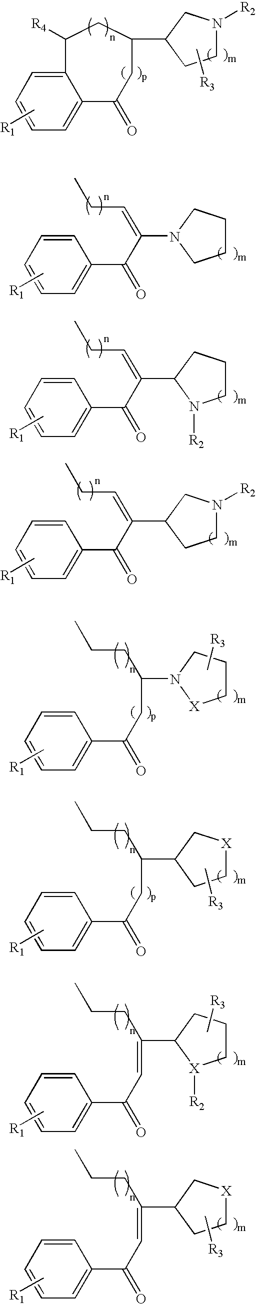

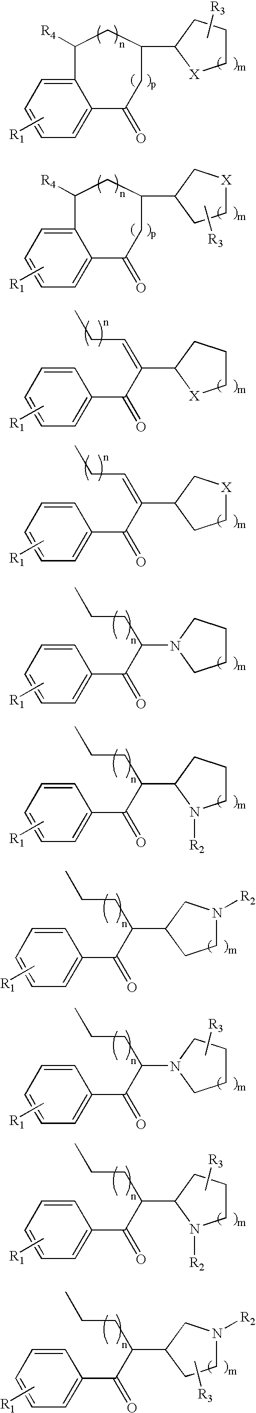

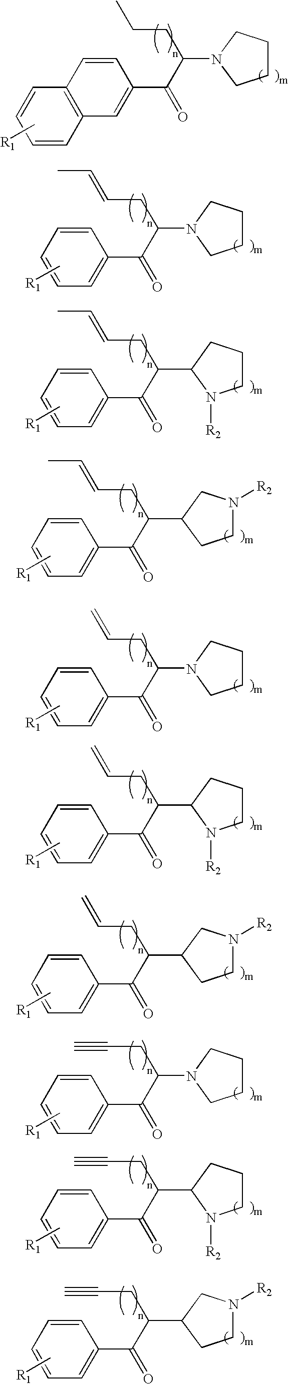

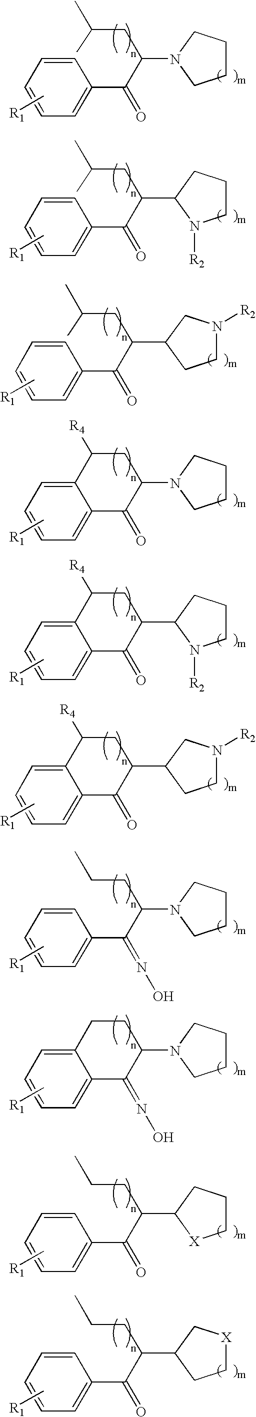

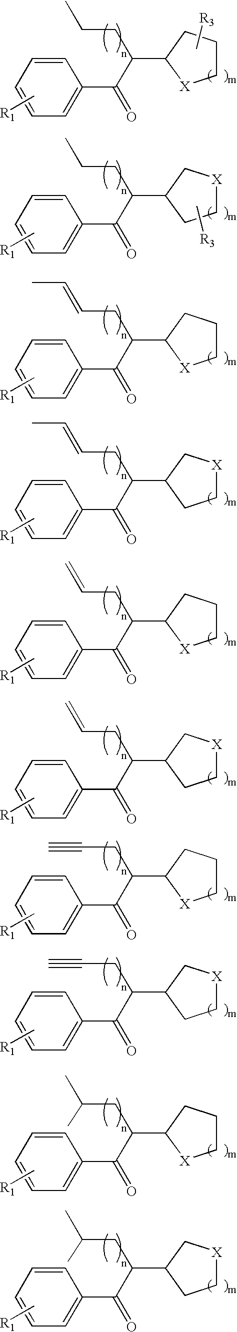

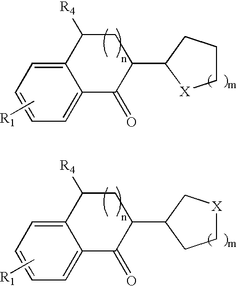

wherein,
R=one to four substituents independently selected from the group consisting of H, halogen (preferably F, Br, Cl, or I), substituted or unsubstituted alkyl (preferably methyl, ethyl, isopropropyl, isobutyl, or t-butyl), substituted or unsubstituted alkoxy (preferably methoxy), substituted or unsubstituted alkenyl, substituted or unsubstituted alkenyloxy, substituted or unsubstituted alkynyl, substituted or unsubstituted alkynyloxy, (CH₂)ₙ—Ar, OH, OC(O)-alkyl (preferably methyl); CF₃; NO₂; NH₂; CN; NHCOCH₃; CO-alkyl (more preferably COCH₃), CH₂OH, (CH₂)ₙOR₂ (in which n is 1 to 4) and (CH₂)ₙOCOR₂; (in which n is 1 to 4);
R₂═H, substituted or unsubstituted alkyl (preferably methyl, ethyl, isopropropyl, isobutyl, or t-butyl), substituted or unsubstituted alkoxy (preferably methoxy), substituted or unsubstituted alkenyl, substituted or unsubstituted alkenyloxy, substituted or unsubstituted alkynyl, substituted or unsubstituted alkynyloxy, or CH₂ArR₁;
R₃=one or two substituents independently selected from the group consisting of H, halogen (preferably F, Br, Cl, or I), substituted or unsubstituted alkyl (preferably methyl, ethyl, isopropropyl, isobutyl, or t-butyl), substituted or unsubstituted alkoxy (preferably methoxy), substituted or unsubstituted alkenyl, substituted or unsubstituted alkenyloxy, substituted or unsubstituted alkynyl, substituted or unsubstituted alkynyloxy, OH, (CH₂)ₙAR₁; CF₃; NO₂; NH₂; CN; NHCOCH₃, CO-alkyl (preferably COCH₃), CH₂OH, (CH₂)ₙOR₂ (in which n is 1 to 4) and (CH₂)ₙOCOR₂; (in which n is 1 to 4);
R₄═H, halogen (preferably F, Br, Cl, or I), substituted or unsubstituted alkyl (preferably methyl), substituted or unsubstituted alkoxy (preferably methoxy), substituted or unsubstituted alkenyl, substituted or unsubstituted alkenyloxy, substituted or unsubstituted alkynyl, substituted or unsubstituted alkynyloxy, OH, OC(O)-alkyl (preferably methyl); CF₃; NO₂; NH₂; CN; NHCO-alkyl (preferably NHCOCH₃), COCH₃, CH₂OH, (CH₂)ₙOR₂ (in which n is 1 to 4) and (CH₂)ₙOCOR₂; (in which n is 1 to 4);
Ar is an aromatic group (preferably phenyl or naphthyl);
n=0-4;
m, p=0-2; and
X═O, CH₂, S, SO₂, or SO; or a pharmaceutically acceptable salt thereof;
   with the proviso that, when the compound is a racemic mixture, the compound is not α-pyrrolidino-valerophenone, pyrovalerone, 1-phenyl-2-pyrrolidino-3-methylbutan-1-one, 1-(p-methoxy-phenyl)-2-pyrrolidino-pentan-1-one, 1-(p-hydroxy-phenyl)-2-pyrrolidino-pentan-1-one, 1-phenyl-2-pyrrolidino-butan-1-one, 1-phenyl-2-pyrrolidino-heptan-1-one, 1-(p-chloro-phenyl)-2-pyrrolidino-pentan-1-one, 1-(m-methyl-phenyl)-2-pyrrolidino-pentan-1-one, 1-phenyl-2-pyrrolidino-nonan-1-one, 1-(p-methoxy-phenyl)-2-pyrrolidino-hexan-1-one, or α-(2 ′-methyl-pyrrolidino)-valerophenone.

In preferred embodiments, R₁ represents F (at the 2, 3 or 4 position); Cl (at the 2, 3 or 4 position); I (at the 2, 3 or 4 position) 3,4-diCl; 3-C1,4-C(CH₂)CH₃; 3-Br,4-isopropyl; 3-I,4-C(CH₂)CH₃; 4-C1,3-C(CH₂)CH₃; 4-Br,3-isopropyl; 4-I,3-isopropyl; 3,4-diOH; 3,4-diOAc; 3,4-diOCH₃; 3-OH,4Cl; 3-OH,4-F; 3-OAc,4-Cl; 3-OAc, 4-F; 3-C1,4-OH; 3-F,4-OH; 3-C1,4-OAc; or 3-F,4-OAc. In certain preferred embodiments, R₁ is an aromatic group.

In certain preferred embodiments, R₁ is selected from the group consisting of methyl, isopropyl, isobutyl, tert-butyl, 3,4-diCl; 3-C1,4-C(CH₂)CH₃; 3-Br,4-C(CH₂)CH₃; 3-I,4-C(CH₂)CH₃; 4-C1,3-C(CH₂)CH₃; 4-Br,3-C(CH₂)CH₃; 4I,3-C(CH₂)CH₃; 3,4-diOH; 3,4-diOAc; 3,4-diOCH₃; 3-OH,4-Cl; 3-OH,4-F; 3-OAc, 4-Cl; 3-OAc, 4-F; 3-Cl,4-OH; 3-F,4-OH; 3-C1,4-OAc; 3-F,4-OAc; and CH₂OH. In more preferred embodiments, R₁ is selected from the group consisting of H, 4-methyl, 3,4-diCl; and 4-Br. In certain preferred embodiments, R₂ is selected from the group consisting of lower alkyl (more preferably methyl and —CH₂-phenyl.

In certain preferred embodiments, R₃ is selected from the group consisting of lower alkyl (more preferably methyl), halogen (more preferably chloro), hydroxyl, and —OCH₃.

In certain preferred embodiments, both m and n are 1.

Certain preferred compounds of the invention are represented by the following structure (Formula I),

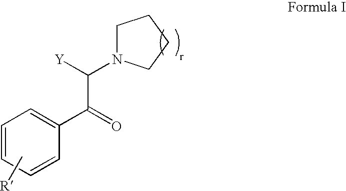

in which R′ represents one to four substituents independently selected from the group consisting of H, halogen (preferably F, Br, Cl, or I), substituted or unsubstituted alkyl (preferably methyl, ethyl, isopropropyl, isobutyl, or t-butyl), substituted or unsubstituted alkoxy (preferably methoxy), substituted or unsubstituted alkenyl, substituted or unsubstituted alkenyloxy, substituted or unsubstituted alkynyl, substituted or unsubstituted alkynyloxy, (CH₂)ₙ—Ar, OH, OC(O)-alkyl (preferably methyl), CF₃, NO₂, NH₂, CN, NHCOCH₃, CO-alkyl (more preferably COCH₃), CH₂OH, (CH₂)ₙOR₂ (in which n is 1 to 4) and (CH₂)ₙOCOR₂ (in which n is 1 to 4); Y is an aliphatic group having from 1 to 8 carbons in a straight, branched (3 to 8 carbon), or cyclic (3 to 8 carbon) aliphatic chain, and r is 1 or 2; provided that, when the compound is a racemic mixture, 1) if Y is n-propyl, and r is 1, then R′ is not H, 4-methyl, 4-hydroxy, 4-methoxy, 4-chloro, or 3-methyl; and 2) if Y is ethyl, isopropyl, n-butyl, n-pentyl, or n-heptyl, and r is 1, then R′ is not H for every occurrence.

Compounds of Formula I may exist either as the racemate or as the substantially enantiomerically pure R— or (most preferably) S-enantiomer (e.g., the 2S enantiomer) at the carbon atom adjacent the ketone functionality. In certain preferred embodiments, R′ is 4-F,4-Br, or 4-I; R′ is 3,4-Cl; R′ is 3,4-OH; R′ is 4-acetamido; R′ is 4-nitro; R′ is 2-methyl; R′ is 3-I; R is 4-hydroxymethyl; R′ is 4-C(O)O-alkyl (most preferably methyl); R′ is 4-alkynyl (more preferably 4-(prop-1-ynyl); or R′ is an aromatic ring attached at the 4-position (more preferably 4-(2′-thienyl), 4-(2′-furyl) or 4-(2′-naphthyl). In more preferred embodiments, R′ is 3,4-dichloro. In certain preferred embodiments, R′ represents 3-OAc, 4-OAc, or 3,4-diOAc (OAc ebbing the group OCOCH₃). In certain preferred embodiments, the aliphatic group is an n-propyl group. In certain preferred embodiments, when the compound is a 2S enantiomer, and the aliphatic chain is an n-propyl group, R′ is H, 4-methyl, 4-methoxy, 4-hydroxy, or 3-methyl. In certain preferred embodiments, the aliphatic chain is an allyl group, most preferably where R is 4-methyl. In certain preferred embodiments, the aliphatic chain is an ethyl group, most preferably where R′ is 3,4-Cl. In certain preferred embodiments, the aliphatic chain is an isobutyl group, most preferably where R′ is 4-methyl. In certain preferred embodiments, r is 2, most preferably when R is 3,4-Cl.

In another embodiment, the invention provides compounds represented by the structure (Formula II)

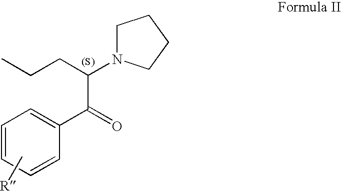

in which R″ represents one to four substituents selected from the group consisting of halogen, lower alkyl, lower alkenyl, lower alkynyl, aryl, —CF₃, hydroxy, nitro, amido (more preferably —NHC(O)-methyl), —(O)CO-alkyl (preferably —(O)CO-methyl) and —C(O)O-alkyl (preferably —C(O)O-methyl; and pharmaceutically acceptable salts thereof. In Formula II, the indication (S) signifies that the compound possesses the 2S configuration. In preferred embodiments of the compound of Formula II, R″ represents 4-alkyl, more preferably 4-methyl. In other preferred embodiments, R″ represents 3,4-dichloro.

In accord with the present invention, novel compounds are provided that bind to monoamine transporters, preferably the DAT. Certain preferred compounds also have a high selectivity for the DAT versus the SERT.

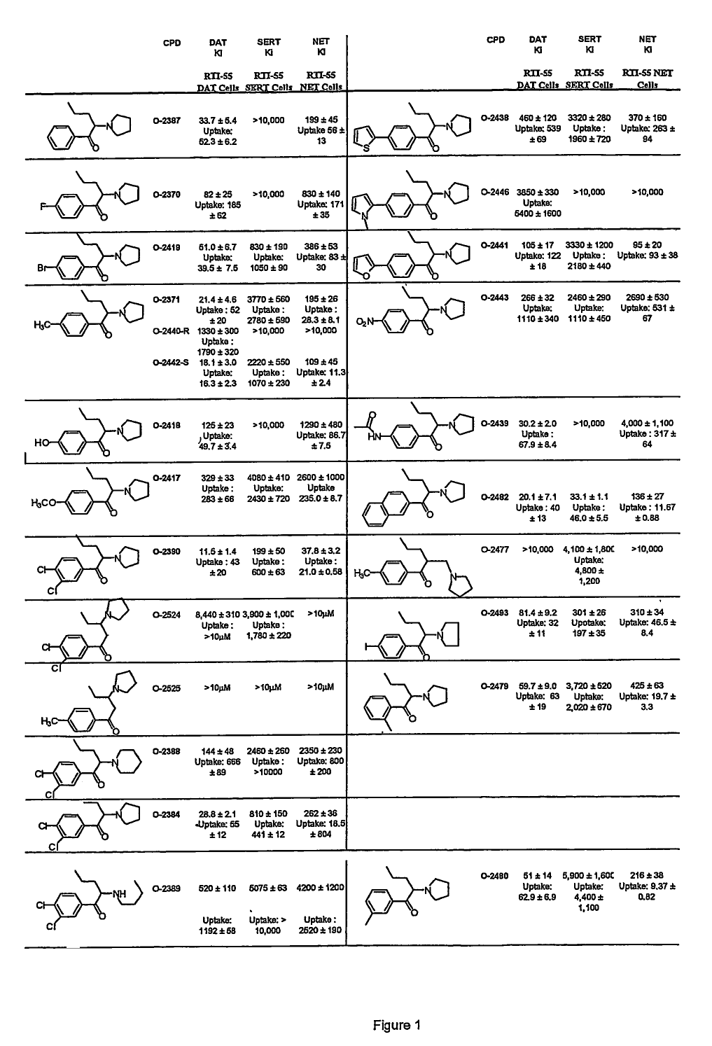

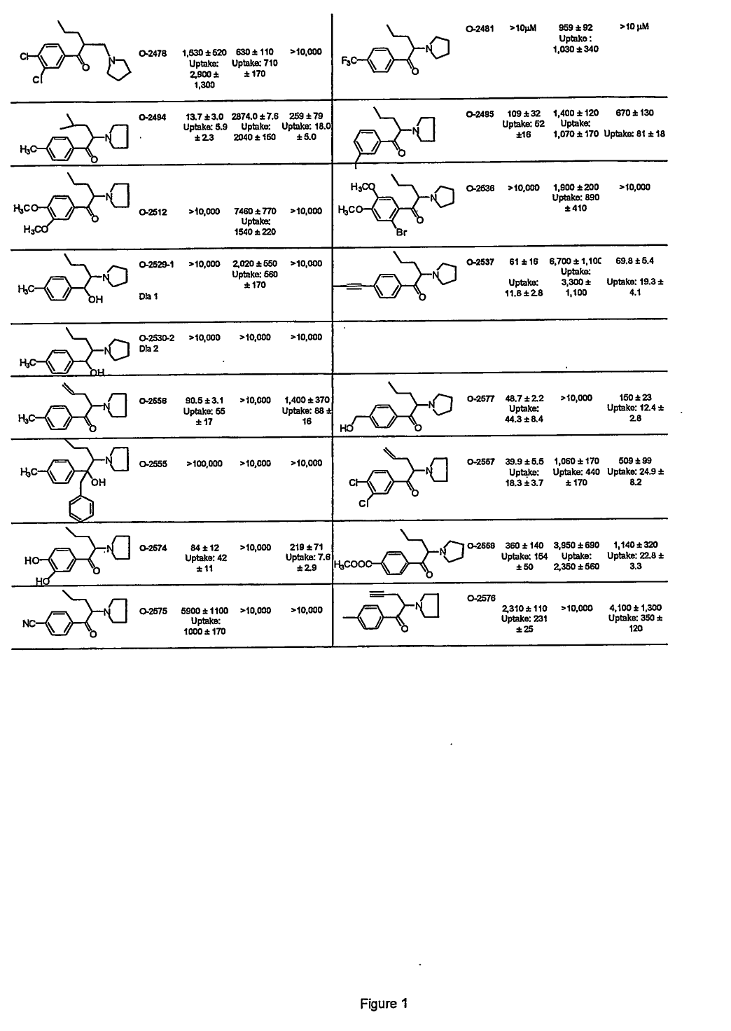

In a preferred embodiment, the novel compounds, for example pyrovalerone analogs are potent and selective DAT inhibitors (see, e.g., Table 2 and FIG. 1). It has now been found that the 2S-enantiomer of pyrovalerone is a more potent DAT inhibitor than the 2R-enantiomer. Accordingly, in certain preferred embodiments, a compound of Formula I is the substantially enantiomerically pure 2S-enantiomer. In certain preferred embodiments, a compound of Formula I is the substantially enantiomerically pure 2R-enantiomer. It has also been found that compounds of Formula I in which R′ represents 3,4-dichloro substitution are unexpectedly desirable; accordingly, in certain preferred embodiments, R′ represents 3,4-dichloro.

Synthesis of these analogs is readily achieved as explained in detail in the examples which follow and exemplified as shown in Scheme I. An energy minimization and overlay was conducted of WIN 35,428 and the 2R and 2S enantiomers of pyrovalerone wherein the pyrrolidine nitrogens and the centroids of the aromatic rings were used as overlay controls. The propyl side chain in the 2S-configuration clearly overlays with the C2-β-carbomethoxy of the tropane. However the 2R-pyrovalerone overlay places the propyl chain in a position similar to that of the 2α-carbomethoxy of the tropane (azabicyclo[3.2.1]octane).

The starting materials, 2, are commercially available or accessible by literature routes from 1 (a substituted benzonitrile) or valerophenone. Bromination (Br₂, AlCl₃) of 2 generally proceeds in high yield and treatment with the secondary amine provides 4 in good yield. Other analogs have alternate aromatic systems, e.g. naphthyl, thiophene or pyrrole, shorter or longer alkyl chains, or are compounds in which the N to aromatic centroid distance has been altered (e.g. 7, 8).

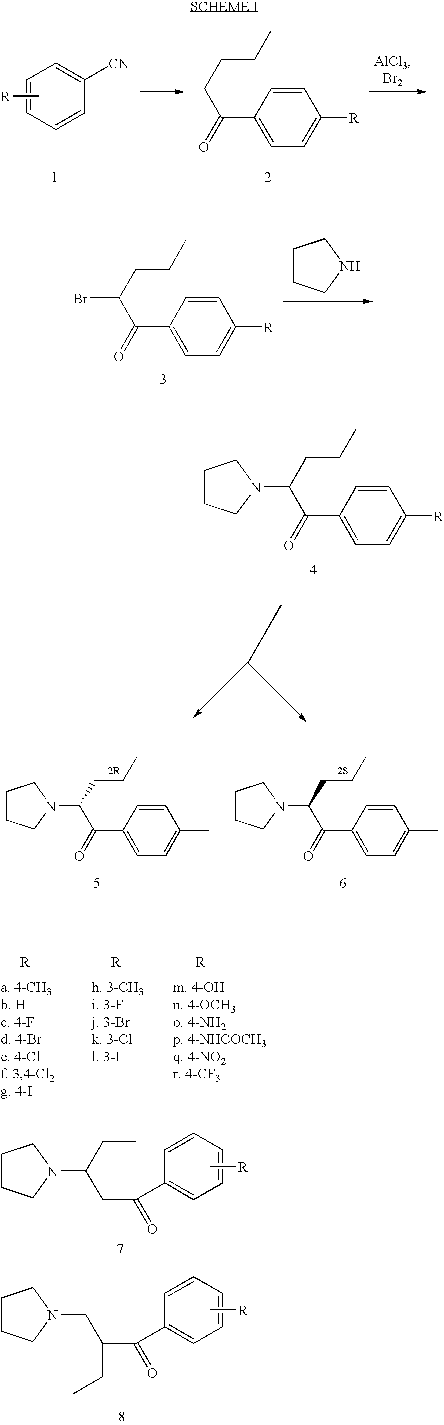

The compounds of the present invention provide a broad array of molecules including compounds that bind with very high affinity. Selectivity for inhibition of the DAT versus the serotonin transporter (SERT) is another property of the compounds of the invention of considerable relevance for development of medications and for probes useful to image the DAT in living brain. Preferred compounds for DAT imaging agents have high DAT:SERT selectivity.

The compounds of the present invention can exhibit extremely potent and selective binding for the DAT, either in vivo or in vitro. Preferred compounds of the present invention exhibit the desired target:non-target (DAT:SERT) specificity. Preferably, the selectivity ratio of binding of SERT to binding of DAT is greater than about 10 (i.e., the compounds bind to DAT with 10-fold greater affinity than to SERT), preferably greater than about 30 and more preferably 50 or more.

In addition, the preferred compounds are potent, preferably having an IC₅₀ for DAT less than about 500 nM, preferably less than 60 nM, more preferably less than about 20 nM, and most preferably less than about 3 nM.

Using the combination of selectivity (SERT/DAT ratio) and potency (IC₅₀) information for these compounds, one of ordinary skill in the art can readily select the appropriate compound for the desired application, e.g., imaging or treatment. The DAT is enantioselective (Reith, M. E. A. et al., Biochem. Pharmocol. 1986, 35, 1123-1129; Ritz, M. C. et al., Science 1987, 237, 1219-1223; Madras, B. K. et al., J. Pharmocol. Exp. Ther. 1989, 251, 131-141; Meltzer, P. C. et al., J. Med. Chem. 1994, 37, 2001-2010; Sershen, H. et al., Neuropharmacology 1980, 19, 1145-1148; Carroll, F. I. et al., J. Med. Chem. 1992, 35, 969-981; Carroll, F. I. et al., in Drug Design for Neuroscience; A. P. Kozikowski, Ed.; Raven Press, Ltd. New York, 1993; 149-166).

The amine-containing compounds of the invention can be prepared either as free bases or-as a pharmacologically active salt thereof such as hydrochloride, tartrate, sulfate, mesylate, naphthalene-1,5-disulfonate or the like (i.e., a pharmaceutically acceptable salt). Additional pharmaceutically acceptable salts are known in the art, and a suitable salt form of the compounds of the invention can be chosen according to such considerations as solubility, crystallinity, ease of synthesis, and the like.

Compounds can be isolated and purified according to a variety of methods known in the art, including chromatography (including HPLC, thin-layer chromatography, and the like), recrystallization, and the like. In certain preferred embodiments, a compound of the invention is at least 70% pure, more preferably at least 80, 90, 95, 98, or 99% pure.

The present invention also provides pharmaceutical compositions, preferably comprising the compounds of the present invention in a pharmaceutically acceptable carrier. Pharmaceutically acceptable carriers are well known to those skilled in the art. An exemplary pharmaceutical composition is a therapeutically effective amount of a compound of the invention optionally included in a pharmaceutically-acceptable and compatible carrier. The term “pharmaceutically-acceptable and compatible carrier” as used herein, and described more fully below, refers to e.g., one or more compatible solid or liquid filler diluents or encapsulating substances that are suitable for administration to a human or other animal. The route of administration can be varied but is principally selected from intravenous, nasal, transdermal and oral routes. For parenteral administration, e.g., it will typically be injected in a sterile aqueous or non-aqueous solution, suspension or emulsion in association with a pharmaceutically-acceptable parenteral carrier such as physiological saline.

The term “therapeutically-effective amount” is that amount of the present pharmaceutical compositions which produces a desired result or exerts a desired influence on the particular condition being treated. Various concentrations may be used in preparing compositions incorporating the same ingredient to provide for variations in the age of the patient to be treated, the severity of the condition, the duration of the treatment and the mode of administration. An effective dose of the compound is typically administered to a patient based on IC₅₀ values determined in vitro or in vivo (e.g., in animal studies).

The term “compatible”, as used herein, means that the components of the pharmaceutical compositions are capable of being commingled with the compounds of the present invention, and with each other, in a manner such that there is no interaction that would substantially impair the desired pharmaceutical efficacy.

Dose of the pharmaceutical compositions of the invention will vary depending on the subject and upon particular route of administration used. Pharmaceutical compositions of the present invention can also be administered to a subject according to a variety well-characterized protocols.

In a preferred embodiment, the pharmaceutical composition is a liquid composition in pyrogen-free, sterilized container or vial. The container can be unit dose or multidose. In certain embodiments, instructions for administration of the pharmaceutical composition to a subject may be included, e.g., as a label for the container or as instructions packaged with the container.

The compounds and pharmaceutical preparations of the present invention can be used to inhibit the %-hydroxytryptamine reuptake of a monoamine transporter, particularly reuptake by the dopamine transporter, serotonin transporter or norepinephrine transporter.

Dysfunction of dopamine neurons has been implicated in several neuropsychiatric diseases. Imaging of the dopamine neurons offers important clinical information relevant to diagnosis and therapeutic treatments. Dopamine neurons produce dopamine, release the neurotransmitter and remove the released dopamine with a dopamine transporter protein. Compounds that bind to the dopamine transporter are effective measures of dopamine neurons and can be transformed into imaging agents for PET and for SPECT imaging (see, e.g., Example 70, infra, for use of PET imaging). In identifying a suitable compound for the dopamine transporter, an essential first step is to measure the affinity and selectivity of a candidate at the dopamine transporter. The affinity can be measured by conducting radioreceptor assays. A radiolabeled marker for the transporter, e.g., (³H)WIN 35,428, is incubated with the unlabeled candidate and a source of the transporter, usually brain striatum. The effect of various concentrations of the candidate on inhibiting (³H)WIN 35,428 binding is quantified. The concentration of the compound that inhibits 50% of (³H)WIN 35,428 bound to the transporter (IC₅₀ value) is used as a measure of its affinity for the transporter. A suitable range of concentrations of the candidate typically is about 1 nM up to about 100 nM, more preferably 1 to 10 nM.

It is also desirable to measure the selectivity of the candidate of the dopamine compared with the serotonin transporter. The serotonin transporter is also detectable in the striatum, the brain region with the highest density of dopamine neurons and in brain regions surrounding the striatum. It is desirable to determine whether the candidate compound is more potent at the dopamine than the serotonin transporter. If more selective (>10-fold), the probe will permit accurate measures of the dopamine transporter in this region of interest or will provide effective treatment modality for the dopamine transporter. Therefore, a measure of probe affinity of the serotonin transport is conducted by assays paralleling the dopamine transporter assays. (³H)Citalopram is used to radiolabel binding sites on the serotonin transporter and competition studies are conducted with the candidate compound at various concentrations in order to generate an IC₅₀ value.

Thus, in one embodiment, the invention provides a method for inhibiting 5-hydroxytryptamine reuptake of a monoamine transporter. The method includes the step of contacting the monoamine transporter with a compound of the invention. The step of contacting can occur, e.g., in vitro, e.g., when a whole cell, cell lysate, or purified enzyme is contacted with a solution of the candidate compound for assay purposes. The step of contacting can also occur in vivo, e.g., by administering the compound to a test subject or to a subject in need of such treatment, under conditions such that the compound contacts a monoamine transporter in vivo.

This invention will be illustrated further by the following examples. These examples are not intended to limit the scope of the claimed invention in any manner. The Examples provide suitable methods for preparing and testing compounds of the present invention. However, those skilled in the art may make compounds of the present invention by any other suitable means. As is well known to those skilled in the art, other substituents can be provided for the illustrated compounds by suitable modification of the reactants. When an enantiomerically enriched form of a compound is desired (i.e., not a racemic mixture), substantially pure enantiomers can be prepared either by a suitable asymmetric synthesis (e.g., according to methods known in the art), or a racemic mixture can be prepared and the enantiomers separated, e.g., using chiral chromatography columns, or by separation using a chiral ligand such as a tartrate (see, e.g., Example 39, infra. A variety of methods of preparing or separating enantiomers are known in the art may be used to prepare substantially enantiomeric pure compounds of the invention, or synthetic precursors of the compounds of the invention.

All exemplified target compounds are fully analyzed (mp, TLC, CHN, GC and/or HPLC) and characterized (¹H NMR, ¹³C NMR, MS, IR) prior to submission for biological evaluation. The affinity of all the compounds for the DAT, SERT and NET are measured. NMR spectra are recorded on a Bruker 100, a Varian XL 400, or a Bruker 300 NMR spectrometer. Tetramethylsilane (“TMS”) is used as internal standard. Melting points are uncorrected and are measured on a Gallenkamp melting point apparatus. Thin layer chromatography (TLC) is carried out on Baker Si 250F plates. Visualization is accomplished with iodine vapor, UV exposure or treatment with phosphomolybdic acid (PMA). Preparative TLC is carried out on Analtech uniplates Silica Gel GF 2000 microns. Flash chromatography is carried out on Baker Silica Gel 40 mM. Elemental Analyses are performed by Atlantic Microlab, Atlanta, Ga. and are within 0.4% of calculated values for each element. A Beckman 1801 Scintillation Counter is used for scintillation spectrometry. 0.1% Bovine Serum Albumin (“BSA”) is purchased from Sigma Chemicals. All reactions are conducted under an inert (N₂) atmosphere.

³H-WIN 35,428 (³H-CFT, 2β-carbomethoxy-3β-(4-fluorophenyl)-N-³H-methyltropane, 79.4-87.0 Ci/mmol) and ³H-citalopram (86.8 Ci/mmol) is purchased from DuPont-New England Nuclear (Boston, Mass.). HPLC analyses are carried out on a Waters 510 system with detection at 254 nm on a Chiralcel OC column (flow rate: 1 mL/min).

TABLE 1

| | | CALCULATED | | | | FOUND | | | |
|---|---|---|---|---|---|---|---|---|---|
| COMPOUND | CALCULATED FOR FORMULA | C | H | N | Cl | C | H | N | Cl |
| 0.2558 | C17H24ClN03 | 62.67 | 7.42 | 4.30 | 10.88 | 62.45 | 7.59 | 4.31 | 10.78 |
| 0.2555 | C23H32ClNO | 73.87 | 8.63 | 3.75 | 9.48 | 73.70 | 8.57 | 3.71 | 9.78 |
| 0-2556 | C16H22ClNO | 63.68 | 7.93 | 5.01 | 12.67 | 63.64 | 7.97 | 5.02 | 12.50 |
| 0-2557 | C15H1SC13NO | 53.83 | 542 | 4.19 | 31.78 | 53.82 | 5.55 | 4.07 | 31.65 |
| 0-2574 | C15H22BrNO3 | 52.34 | 6.44 | 4.07 | 23.21(Br | 52.40 | 6.48 | 4.03 | 22.98 |
| 0-2575 | C16H21ClN20.1/4H2O | 64.64 | 7.29 | 9.42 | 11.92 | 64.74 | 729 | 9.31 | 11.92 |
| 0-2576-1 | C16H2OClNO | 69.18 | 7.26 | 5.04 | 12.76 | 68.91 | 7.36 | 5.05 | 12.97 |
| 0-2577 | C16H24ClN02.1/4H2O | 63.57 | 8.17 | 4.63 | 11.73 | 63.55 | 8.13 | 4.68 | 11.55 |
| 0-2536 | C17H25BRClN03.2/3H2O | 48.76 | 6.34 | 3.34 | 8.47 | 48.65 | 6.28 | 3.33 | 8.44 |
| 0-2529 | C16H226ClNO | 67.71 | 9.23 | 4.93 | 12.49 | 67.70 | 9.26 | 4.91 | 12.55 |
| 0-2537 | C18H24ClNO | 70.69 | 7.91 | 4.58 | 11.59 | 70.45 | 7.96 | 4.59 | 11.81 |
| 0-2512 | C17H26ClN03 | 62.28 | 7.99 | 4.27 | 10.81 | 62.04 | 8.01 | 4.24 | 11.06 |
| 0-2494 | C17H26ClNO | 69.02 | 8.86 | 4.73 | 11.98 | 68.92 | 8.84 | 4.69 | 12.00 |
| 0-2493 | C15H21C11NO | 45.76 | 5.38 | 3.56 | 9.01 | 45.81 | 5.49 | 3.39 | 9.17 |
| 0-2432 | C19H24ClNO | 71.80 | 7.61 | 4.41 | 11.15 | 71.53 | 7.72 | 4.41 | 11.14 |
| 0-2481 | C16H21ClF3NO | 57.23 | 6.30 | 4.17 | 10.56 | 57.12 | 634 | 4.14 | 10.44 |
| 0-2430 | C16H24ClNO | 68.19 | 8.58 | 4.97 | 12.58 | 68.07 | 8.68 | 4.88 | 12.67 |
| 0-2479 | C16H24ClN0.92/100H2O | 64.42 | 8.73 | 4.69 | 11.88 | 64.39 | 8.69 | 4.71 | 11.98 |
| 0-2477 | C17H26ClNO | 69.02 | 8.86 | 4.73 | 11.98 | 68.95 | 8.94 | 4.77 | 12.09 |
| 0-2478 | C16H22C13NO | 54.80 | 6.32 | 3.99 | 30.33 | 54.82 | 636 | 4.06 | 30.39 |
| 0-2446 | C20H27ClN20.2/3H2O | 66.93 | 7.96 | 7.31 | 9.88 | 66.85 | 7.88 | 7.79 | 9.82 |
| 0-2441 | C16H24ClNO | 68.19 | 8.58 | 4.97 | 12.58 | 68.06 | 8.60 | 4.96 | 12.47 |
| 0-2442 | C16H24ClNO | 68.19 | 8.58 | 4.97 | 12.58 | 68.24 | 8.62 | 4.99 | 12.48 |
| 0-2438 | C19H24ClN05 | 65.22 | 6.91 | 4.00 | 10.13 | 65.11 | 6.77 | 3.96 | 9.99 |
| 0-2441 | C19H24ClN02 | 63.36 | 7.25 | 4.20 | 10.62 | 68.11 | 7.17 | 4.21 | 10.67 |
| 0-2443 | C15H21C1N203.0.42H20.0.08HCl | 55.72 | 6.83 | 8.66 | 11.88 | 55.73 | 6.80 | 8.48 | 11.91 |
| 0-2439 | C17H25ClN202.1/2H2O | 61.16 | 7.85 | 8.39 | 10.62 | 61.32 | 7.70 | 8.40 | 10.68 |
| 0-2419 | C15H21BrClNO | 51.97 | 6.11 | 4.04 | 10.23 | 51.78 | 6.00 | 3.95 | 10.28 |
| 0-2418 | C1SH22ClN02 | 63.48 | 7.81 | 4.94 | 12.49 | 63.43 | 7.90 | 5.00 | 12.30 |
| 0-2417 | C16H24ClN02.1/2H20.1/2HCl | 59.12 | 7.91 | 4.31 | 16.36 | 59.39 | 8.07 | 4.36 | 16.22 |
| 0-2530 | C16H26ClNO | 67.71 | 9.23 | 4.93 | 1249 | 67.47 | 9.29 | 4.94 | 12.56 |
| 0-2539 | C13H18ClNO | 65.13 | 7.57 | 5.84 | 14.79 | 65.30 | 7.62 | 5.83 | 14.85 |
| 0-2538 | C12H14Cl3NO | 48.92 | 4.79 | 4.75 | 36.10 | 48.91 | 4.77 | 4.67 | 36.02 |
| 0-2511 | C17H25ClN0.38/100H2O | 67.48 | 8.91 | 4.63 | 11.72 | 67.40 | 892 | 4.61 | 11.54 |
| 0-2525 | C16H24ClNO | 68.19 | 8.58 | 4.97 | 12.58 | 68.11 | 8.55 | 5.01 | 12.70 |
| 0-2524 | C1SH2OCl3NO.1/3H2O | 52.57 | 6.08 | 4.09 | 31.04 | 52.40 | 5.98 | 4.18 | 31.28 |
| 0-2495 | C15H21C11NO | 45.76 | 5.38 | 3.56 | 9.01 | 45.65 | 5.37 | 3.5 | 8.88 |
| 0-2390 | C15H2OC13NO | 53.51 | 5.99 | 4.16 | 31.59 | 53.37 | 5.93 | 4.14 | 31.65 |
| 0-2389 | C15H22C13NO | 53.19 | 6.55 | 414 | 31.4 | 5313 | 6.48 | 4.12 | 31.55 |
| 0-2388 | C16H22C13NO | 54.80 | 6.32 | 3.99 | 30.33 | 54.62 | 6.34 | 4.08 | 30.52 |
| 0-2387 | C1SH22ClNO | 67.28 | 8.28 | 5.23 | 13.24 | 67.50 | 8.35 | 5.18 | 13.12 |
| 0-2370 | C15H21ClFNO | 63.04 | 7.41 | 4.90 | | 63.32 | 745 | 4.85 | |
| 0-2384 | C14H18C13NO | 52.11 | 5.62 | 4.34 | | 52.14 | 5.55 | 4.26 | |
| 0-2371 | C16H24ClNO.1/6H2O | 67.47 | 861 | 4.92 | | 67.47 | 856 | 4.91 | |

### EXAMPLES

### Materials and Methods

Compounds were prepared employing the same method, General Procedure A as illustrated by Scheme I, except where noted.

General Procedure A: α-Bromoketone (10 mmol) was dissolved in Et₂O (10 mL) (EtOH is a suitable alternative solvent) and cooled on an ice bath. Pyrrolidine (22 mmol) was added all at once. The mixture became orange and an oil was observed to separate from the solution. After 1-24 h stirring at room temperature, the crude reaction mixture was partitioned between H₂O (10 mL) and Et₂O. The Et₂O layer was separated and the aqueous layer was washed with Et₂O (2×10 mL). The ether layer was extracted with I M aqueous HCI (2×10 mL), then back-extracted into Et₂O (3×10 mL) by basification to pH 8-9 with 20% aqueous Na₂CO₃. The Et₂O extracts were dried (MgSO₄) and filtered. The filtrate was treated with 2 M ethereal HCI (usually 5-10 mL) until precipitation of solid or oil had ceased. Solids (oils were triturated to give solids) were collected by filtration and recrystallized from EtOH/Et₂O.

### Example 1

1-(3,4-Dihydroxy-phenyl)-2-pyrrolidin-1-yl-pentan-1-one, hydrogen bromide salt. 1-(3,4-Dimethoxyphenyl)-2-pyrrolidin-1-yl-pentan-1-one (1.50 g, 4.6 mmol) was freed from its hydrogen chloride salt by treatment with aqueous Na₂CO₃ and extracting into CH₂CI₂. The organics were dried (MgS0₄), filtered, and reduced to a pale yellow oil in vacuo. The oil was taken up in CH₂Cl₂ (10 mL) and cooled to −78 ° C., whereon BBr₃ (46 mL, 1.0 M solution in CH₂Cl₂, 46 mmol) was added dropwise over 0.5 h. The resulting yellow mixture was warmed slowly to room temperature and stirred for 3 h. The yellow solution was hydrolyzed cautiously by addition of aq. Na₂CO₃ (20% solution) until the pH was 8, then water (50 mL) was added and the solution was allowed to stand overnight. Neutral organics were extracted from the mixture by separation of the CH₂Cl₂ layer which was then discarded. The aqueous layer was acidified to pH 3 with 1 M HCI, most of the water was removed by rotary evaporation, and the remaining volume of ca 10 mL was allowed to cool in the refrigerator. After 3 d, a white solid separated from the solution and was collected by filtration. Recrystallization (EtOH/Et₂O) afforded pure 1-(3,4-dihydroxyphenyl)-2-pyrrolidin-1-yl-pentan-1-one (0.60 g, 44%) as its hydrogen bromide salt, an off-white solid; Mp 181-182° C.; ¹H NMR δ 10.42 (s, 1H), 10.1-9.9 (br, 1H), 9.59 (s, 1H), 7.51 (dd, I H), 7.43 (d, 1H), 6.91 (d, 111), 5.35-5.25 (br, 111), 3.75-3.5 (br, 1H). 3.5-3.3 (br, I H), 3.3-3.15 (br, 1H), 3.0-2.85 (br, 1H), 2.1-1.8 (m, 6H), 1.3-1.0 (m, 2H), 0.80 (t, J=7 Hz, 3H); ¹³C NMR δ 194.8, 153.4, 146.4, 126.7, 123.5, 116.0, 115.9, 675, 54.5, 52.3, 32.8, 23.2, 17.9, 14.3; APCI MS m/z 264 (M+1); Anal. (C₁₅H₂₂BrNO₃) C, H, N, Br.

### Example 2

4-(2-Pyrrolidin-1-yl-pentanoyl)-benzonitrile, hydrogen chloride salt. This compound was prepared, in 70% yield, as described in General Procedure A, with slight modifications; Mp 197-199° C. (dec.); ¹H NMR δ 10.9-10.7 (br, 1H), 8.24 (d, 2H), 8.14 (d, 2H), 5.7-5.55 (br, m, 1H), 3.7-3.6 (br, m, 1H), 3.6-3.5 (br, m, 1 H), 3.3-3.1 (br, m, 2H), 2.1-1.8 (m, 6H), 1.4-1.2 (m, 1 H), 1.1-0.9 (m, 1 H), 0.77 (t, J=7 Hz, 3H); ¹³C NMR δ 196.2, 137.5, 133.2, 129.4, 117.9, 116.6, 67.8, 53.7, 51.9, 31.3, 22.9, 17.2, 13.7; APCI MS m/z 257 (M+1); Anal. (C₁₆H₂₁ClN₂O.¼H₂0) C, H, N, Cl.

### Example 3

2-Pyrrolidin-1-yl-1 p-tolyl-pent-4-yn-1-one, hydrogen chloride salt. 2-Pyrrolidin-1-yl-1-p-tolyl-ethanone, (25 g, 104 mmol) was freed from its hydrogen chloride salt by treatment with aqueous Na₂CO₃ and extraction into Et₂O. The organics were dried (MgSO₄), filtered and reduced in vacuo to a yellow oil. This oil was taken up in toluene (200 mL), and NaNH₂ was added to the stirring solution which was subsequently heated to approximately 120° C. (oil bath temperature) for 0.5 h. Propargyl bromide (13 mL, 80% w/w solution in toluene, 14 g, 115 mmol) was added to the resulting cooled (oil bath temperature at approximately 100° C.) orange mixture at such a rate that steady reflux was allowed to occur with concomitant NH₃ evolution. Upon complete addition (0.5 h), the mixture was cooled slowly to room temperature and was then hydrolyzed cautiously by addition of water (100 mL). The toluene layer was separated and the aqueous layer was extracted with toluene (2×50 mL). The combined organics were dried (MgS0₄), filtered and reduced in vacuo to a brown oil that was taken up in Et₂O (50 mL). 2 M HCl in Et₂O was added to the ethereal solution of the oil. Trituration afforded a brown solid attempted recrystallization of which, from EtOH/Et₂O gave an impure brown oil. The solvents were removed by rotary evaporation and the free base was prepared by addition of 2 M NaOH solution until pH 8-9. The organics were extracted into Et₂0 (3×100 mL) to give a light brown solution. Back-extraction into 1 M HCl (3×50 mL) gave a light yellow solution. The water was removed by rotary evaporation, then lyophilization to give 5.3 g of a light brown gum. Recrystallization from EtOH/Et₂O afforded pure 2-pyrrolidin-1-yl-1 p-tolyl-pent-4-yn-1-one, as its hydrogen chloride salt (3.15 g, 11%): Mp 178° C. (dec.); ¹H NMR δ 10.6-10.4 (br, 1H), 7.97 (d, 2H), 7.45 (d, 2H), 5.66 (m, 1H), 3.7-3.2 (m, 3H), 3.2-2.9 (m, 4H), 2.43 (s, 3H), 2.1-1.8 (m, 4H); ¹³C NMR δ 193.9, 146.0, 131.1, 129.7, 129.2, 76.8, 76.6, 65.2, 54.0, 52.0, 22.9, 22.9, 21.3, 20.0; APCI MS m/z 242 (M+1); Anal. (C₁₆H₂₀ClNO) C, H, N, Cl.

### Example 4

1-(4-Hydroxymethyl-phenyl)-2-pyrrolidin-1-yl-pentan-1-one, hydrogen chloride salt. This compound was prepared, in 79% yield, as described in General Procedure A, with slight modifications; Mp 186-187° C. (dec.); ¹H NMR δ 10.6-10.4 (br, 1H), 8.05 (d, 2H), 7.56 (d, 2H), 5.7-5.4 (br, m, 2H), 4.62 (s, 2H), 3.7-3.55 (j, 1 H), 3.55-3.3 (m, 1 H), 3.35-3.15 (m, 1 H), 3.1-3.0 (m, 1 H), 2.1-1.8 (m, 6H), 1.3-1.15 (m, 1H), 1.15-0.95 (m, 1H), 0.78 (t, J=7 Hz, 3H); ¹³C NMR δ 196.2, 150.4, 132.8, 128.8, 126.7, 67.4, 62.2, 53.8, 51.9, 31.8, 22.8, 17.3, 13.7; MS 262; Anal. (C₁₆H₂₄CINO₂.¼H₂0) C, H, N, Cl.

### Example 5

1-Phenyl-3-pyrrolidin-1-yl-2 p-tolyl-hexan-2-ol, hydrogen chloride salt. The pyrovalerone (2.0 g, 7.1 mmol) was freed from its HCI salt by treatment with 20% Na₂CO₃ and extraction of the organics into Et₂O. The Et₂O extracts were dried (MgS0₄), filtered and reduced in vacuo to a pale yellow oil. This oil was taken up in toluene (20 mL) and cooled on an ice bath. Benzylmagnesium chloride (3.9 mL, 2.0 M solution in THF, 7.8 mmol, 1.1 mol eq.) was added via syringe over 5 min to the solution which was subsequently hydrolyzed by addition of 1 M HCI (20 mL). The resulting flocculent white precipitate was collected by filtration, washed with 1 M HCI (5 mL), then Et₂O (50 mL), dried under suction, then in air. Recrystallization from EtOH/Et₂O afforded pure 1-phenyl-3-pyrrolidin-1-yl-2-p-tolyl-hexan-2-ol, as its hydrogen chloride salt (2.0 g, 75%): Mp 211° C. (dec.); ¹H NMR δ 9.5-9.3 (br, 1H), 7.41 (d, 2H), 7.2-7.0 (m, 7H), 6.07 (s, 1H), 3.85-3.6 (br, m, 2H), 3.41 (m, 2H), 3.15-2.9 (m, 2H), 3.8-3.6 (m, 1H), 2.25 (s, 3H), 1.95-1.75 (br, m, 5H), 1.4-1.1 (m, 2H), 1.1-0.9 (m, 1H), 0.78 (t, 311); ¹³C NMR δ 137.7, 136.4, 136.2, 130.8, 128.3, 127.3, 126.7, 125.8, 77.6, 72.0, 55.9, 44.0, 26.3, 24.4, 22.6, 22.2, 20.6, 14.0; APCI MS m/z 338 (M+1); Anal. (C₂₃H₃₂ClNO) C, H, N, Cl.

### Example 6

2-Pyrrolidin-1-yl-1 p-tolyl-pent-4-ene-1-one, hydrogen chloride salt. This compound was prepared as described above; Mp 196° C. (dec.); ¹H NMR δ 10.8-10.6 (br, 1H), 7.96 (d, 2H), 7.43 (d, 2H), 5.8-5.6 (m, 2H), 5.03 (s, 1H), 5.00 (m, 1H), 3.75-3.6 (br, 1H), 3.6-3.4 (br, I H), 3.4-3.2 (br, m, 1H), 3.15-3.0 (br, m, 1H), 3.85-3.65 (br, m, 2H), 2.42 (s, 3H), 2.2-1.85 (br, m, 4H); ¹³C NMR δ 195.2, 145.8, 131.8, 130.6, 129.7, 129.0, 120.1, 66.9, 53.8, 52.0, 34.2, 22.9, 21.3; APCI MS m/z 244 (M+1); Anal. (C₁₆H₂₂ClNO) C, H, N, Cl.

### Example 7

1-(3,4-Dichloro-phenyl)-2-pyrrolidin-1-yl-pent-4-ene-1-one, hydrogen chloride salt. This compound was prepared as described above; Mp 176° C. (dec.); ¹H NMR δ 10.8-10.6 (br, 1H), 8.29 (d, 1 H), 8.00 (dd, 1 H), 7.94 (d, 1 H), 5.8-5.6 (m, 2H), 5.07 (s, 1 H), 5.02 (m, 1 H), 3.75-3.6 (br, m, 1 H), 3.6-3.3 (br, m, 1H), 3.3-3.1 (br, m, 2H), 2.77 (m, 2H), 2.2-1.8 (br, m, 4H), ¹³C NMR δ 194.2, 137.8, 134.4, 132.2, 131.6, 130.8, 130.3, 128.8, 120.6, 67.2, 53.9, 52.1, 33.8, 22.9; APCI MS m/z (relative intensity): 302 ((M+1), 100%), 300,298; Anal. (C₁₅H₁₈Cl₃NO) C, H, N, Cl.

### Example 8

4-(2-Pyrrolidin-1-yl-pentanoyl)-benzoic acid methyl ester, hydrogen chloride salt. This compound was-prepared, in 77% yield, as described in General Procedure A, with slight modifications; Mp 202° C. (dec.); ¹H NMR δ 10.7-10.5 (br, 1H), 8.3-8.1 (m, 4H), 5.58 (m, 1H), 3.91 (s, 3H), 3.7-3.5 (br, m, 2H), 3.3-3.05 (br, m, 2H), 2.15-2.85 (br, m, 6H), 1.4-1.2 (m, 1H), 1.15-0.95 (m, 1H), 0.77 (t, J=7 Hz, 3H); ¹³C NMR δ 196.5, 165.3, 137.6, 134.6, 129.8, 129.2, 67.9, 53.9, 52.7, 51.9, 31.4, 22.9, 17.2, 13.7; APCI MS m/z (relative intensity): 290 ((M+1), 100%), 275; Anal. (C₁₇H₂₄ClN0₃) C, H,N, Cl.

### Example 9

0-2536 1-(2-Bromo-4,5-dimethoxy-phenyl)-2-pyrrolidin-1-yl-pentan-1-one, hydrogen chloride salt. This compound was prepared, in 68% yield, as described in General Procedure A, however, the final compound, which contained residual Et₂O that could not be removed by further recrystallization, was dissolved in H₂O and lyophilized; Mp 100-120° C. (dec.); ¹H NMR δ 10.6-10.4 (br, 1H), 7.59 (s, 1H), 7.35 (s, 1H), 5.58 (br, 1 H), 3.89 (s, 6H), 3.7-3.55 (br, 2H), 3.3-3.15 (br, m, 2H), 2.15-1.7 (m, 6H), 1.4-1.2 (m, 1 H), 1.2-1.0 (m, 1H), 0.79 (t, J=7 Hz, 3H); ¹³C NMR δ 196.2, 152.5, 147.9, 127.3, 117.7, 113.7, 112.2, 69.4, 56.6, 56.3, 51.7, 31.2, 22.9, 17.2, 13.7; APCI MS m/z 372, 370 (Br2) (M+1); Anal. (C₁₇H₂₅BrClN0₃.⅔H₂0) C, H, N, Cl.

### Example 10

Compound 0-2529 and Compound 0-2530—2-Pyrrolidin-1-yl p-tolyl-pentan-1-ol, hydrogen chloride salt and 2-Pyrrolidin-1-yl p-tolyl-pentan-1-ol, hydrogen chloride salt. (DIASTEREOISOMER 2-0-2530). Pyrovalerone, hydrogen chloride salt (1.50 g, 5.32 mmol) was suspended in THF (20 mL). LiAIH₄ (0.20 g, 5.3 mmol) was added in several small portions at room temperature to the stirring mixture with slight heat evolution. The resulting clear solution was hydrolyzed cautiously with H₂O, then made acidic by addition of 1M aqueous HCI. The aqueous extracts were collected and basified to pH 8-9 with 20% aqueous Na₂CO₃. The organics were extracted into Et₂0, dried (MgSO₄), filtered, and reduced to an oil in vacuo. Chromatography (5% NEt₃/15% EtOAc/80% hexanes) gave the two diastereoisomers. The hydrogen chloride salts were prepared from 2M ethereal HCl and recrystallized from EtOH/Et₂O to afford 2-Pyrrolidin-1-yl p-tolyl-pentan-1-ol, hydrogen chloride salt (DIASTEREOISOMER 1, 0-2529), a colorless crystalline solid (0.57 g, 37%); Mp 140-142° C.; ¹H NMR δ 10.15-10.0 (br, 1 H), 7.32 (d, 2H), 7.19 (d, 2H), 6.20 (d, J=5 Hz, 1 H), 5.24 (s, 1 H), 3.75-3.65 (br, m, 1H), 3.65-3.5 (br, m, 111), 3.4-3.3 (br, 2H), 3.2-3.05 (br, m, 1H), 2.30 (s, 3H), 2.1-1.8 (br, m, 4H), 1.75-1.6 (m, 1H), 1.4-1.25 (br, m, 1H), 1.1-0.95 (m, 1H), 0.8-0.6 (m, 1H), 0.57 (t, J=7 Hz, 3H); ¹³C NMR δ 36.2, 128.6, 125.5, 69.3, 68.1, 51.5, 26.5, 22.7, 22.5, 20.7, 20.3, 13.7; APCI MS m/z 248 (M+1); Anal. (C₁₆H₂₆ClNO) C, H, N, Cl. and 2-Pyrrolidin-1-yl p-tolyl-pentan-1-ol, as its hydrogen chloride salt, a colorless microcrystalline solid (159 mg, 10%) (DIASTEREOISOMER 2-0-2530, this was the more polar material also); Mp 219° C. (dec.); ¹H NMR δ 9.8-9.65 (br, 1H), 7.33 (d, 2H), 7.20 (d, 2H), 6.53 (d, J=4 Hz, I H), 4.65 (dd J=4.9 Hz, 1H), 3.55-3.3 (m, 3H), 3.3-3.15 (br, m, 1H), 3.15-2.95 (br, m, I H), 2.31 (s, 3H), 2.0-1.85 (br, 4H), 1.55-1.35 (br, m, 2H), 1.05-0.85 (m, 1H), 1.75-1.6 (m, 4H); ¹³C NMR δ 138.4, 137.3, 128.9, 127.1, 72.1, 67.0, 40.3, 40.1, 27.6, 23.3, 23.0, 20.8, 20.0, 13.6; APCI MS m/z 248 (M+1); Anal. (C₁₆H₂₆ClNO) C, H, N, Cl.

### Example 11

Compound 0-2537 1-(4-Propynyl-phenyl)-2-pyrrolidin-1-yl-pentan-1-one, hydrogen chloride salt. 1-(4-Iodo-phenyl)-2-pyrrolidin-1-yl-pentan-1-one, hydrogen chloride salt (500 mg, 1.27 mmol) was taken up in Et₂NH (10 mL) and degassed by purging with N₂. [PdCl₂(PPh₃)₂] (18 mg, 2.5.10⁻⁵ mol) and Cul (2.4 mg, 1.3.10⁻⁵ mol) were added to the stirring solution at room temperature. Propyne was then bubbled through the resulting yellow mixture for 7 h. The mixture was filtered and reduced to an oil in vacuo. The oil was taken up in Et₂O and extracted into 1M aqueous HCI, then back-extracted into Et₂O by treatment with 20% aqueous Na₂CO₃ until pH 8-9. The organic extracts were dried (MgSO₄), filtered, and reduced to a pale yellow oil in vacuo. The hydrogen chloride salt was prepared from 2M ethereal HCI and recrystallized twice from EtOH/Et₂O to give pure 1-(4-Propynyl-phenyl)-2-pyrrolidin-1-yl-pentan-1-one, as a colorless crystalline solid (260 mg, 67%). Mp 231° C. (dec.); ¹H NMR δ 10.6-10.4 (br, IH), 8.04 (d, 2H), 7.62 (d, 2H), 5.55-5.4 (br, m, 1H), 3.7-3.55 (br, 1H), 3.55-3.4 (br, 111), 3.3-3.1 (br, m, 114), 3.1-2.95 (br, m, I H), 2.12 (s, 3H), 2.1-1.8 (br, m, 6H), 1.3-1.15 (m, 1H), 1.15-0.95 (m, 1H), 0.78 (t, J=7 Hz, 3H); ¹³C NMR δ 195.9, 133.1, 131.9, 129.9, 129.1, 92.1, 79.0, 67.5, 53.8, 51.9, 31.7, 22.8, 17.2, 13.7, 4.1; APCI MS m/z 270 (M+1); Anal. (C₁₈H₂₄ClNO) C, H, N, Cl.

### Example 12

Compound 0-2512 1-(3,4-Dimethoxy-phenyl)-2-pyrrolidin-1-yl-pentan-1-one, hydrogen chloride salt. This compound was prepared, in 74% yield, as described in General Procedure A, with slight modifications; Mp 177° C. (dec.); ¹H NMR δ 10.5-10.3 (br, 1H), 7.78 (d, IH), 7.53 (d, 1H), 7.18 (d, 1H), 5.55-5.4 (br, m, 1H), 3.90 (s, 3H), 3.86 (s, 3H), 3.7-3.55 (br, m, 1H), 3.5-3.3 (br, m, 1H), 3.3-3.15 (br, m, 1H), 3.05-2.9 (br, m, 1H), 2.1-1.8 (m, 6H), 1.3-1.0 (m, 2H), 0.80 (t, J=7 Hz, 3H); ¹³C NMR δ 194.7, 154.7, 149.0, 127.2, 124.6, 111.2, 110.5, 66.7, 56.0, 55.7, 53.7, 51.8, 32.1, 22.8, 17.4, 13.7; APCI MS m/z 292 (M+1); Anal. (C₁₇H₂₆ClN0₃) C, H, N, Cl.

### Example 13

Compound 0-2494 4-Methyl-2-pyrrolidin-1-yl-1 p-tolyl-pentan-1-one, hydrogen chloride salt. This compound was prepared, in 68% yield, as described in General Procedure A, with slight modifications; Mp 218° C. (dec.); ¹H NMR δ 10.9-10.75 (br, 1H), 8.06 (d, 2H), 7.45 (d, 2H), 5.46 (m, 1 H), 3.75-3.6 (br, 1 H), 3.6-3.4 (br, 1 H), 3.3-3.0 (br, m, 2H), 2.42 (s, 3H), 2.1-1.7 (m, 6H), 1.45-1.3 (m, 1 H), 0.82 (dd, J=2, 6 Hz, 6H); ¹³C NMR δ 197.2, 164.0, 132.9, 129.9, 129.0, 64.4, 52.7, 51.2, 24.2, 23.3, 22.8, 21.5, 21.3; APCI MS m/z 260 (M+1); Anal. (C₁₇H₂₆CIN0) C, H, N, Cl.

### Example 14

Compound 0-2493 1-(4-Iodo-phenyl)-2-pyrrolidin-1-yl-pentan-1-one, hydrogen chloride salt. This compound was prepared, in 37% yield, as described in General Procedure A, with slight modifications; Mp 218° C. (dec.); ¹H NMR δ 10.75-10.65 (br, 111), 8.05 (d, 2H), 7.84 (d, 2H), 5.53 (m, 1H), 3.7-3.65 (br, 1H), 3.65-3.5 (br, m, 1H), 3.3-3.15 (br, m, I H), 3.15-3.0 (br, m, 1H), 2.1-1.8 (br, m, 6H), 1.35-1.15 (m, I H), 1.15-0.95 (m, 1H), 0.78 (t, J=7 Hz, 3H); ¹³C NMR δ 196.3, 138.2, 133.6, 130.3, 104.6, 67.3, 53.7, 51.9, 31.6, 22.9, 17.3, 13.7; APCI MS m/z 358 (M+1); Anal. (C₁₅H₂₁ClINO) C, H, N, Cl.

### Example 15

Compound 0-2482 1-Naphthalen-2-yl-2-pyrrolidin-1-yl-pentan-1-one, hydrogen chloride salt. This compound was prepared, in 51% yield, as described in General Procedure A, with slight modifications; Mp 221-223° C. (dec.); ¹H NMR δ 10.8-10.6 (br, 1H), 8.92 (s, 1H), 8.2-8.0 (m, 4H), 7.75 (dt, 2H), 5.73 (m, 1H), 3.75-3.6 (br, 1H), 3.6-3.4 (br, m, 1H), 3.35-3.1 (br, m, 2H), 2.2-1.8 (m, 6H), 1.4-1.2 (m, 1H), 1.2-1.0 (m, 1 H), 0.78 (t, J=7 Hz, 3H); ¹³C NMR δ 196.6, 135.7, 132.0, 131.8, 131.7, 129.9, 129.7, 129.0, 127.8, 127.5, 123.4, 67.3, 53.6, 52.0. 31.9, 22.9, 17.4, 13.7; APCI MS m/z 282 (M+1); Anal. (C₁₉H₂₄CINO) C, H, N, Cl.

### Example 16

Compound 0-2481 2-Pyrrolidin-1-yl-1-(4-trifluoromethyl-phenyl)-pentan-1-one, hydrogen chloride salt. This compound was prepared, in 44% yield, as described in General Procedure A, with slight modifications; Mp 228° C. (dec.); ¹H NMR; δ 10.8-10.6 (br, 1H), 8.28 (d, 2H), 8.03 (d, 2H), 5.62 (m, 1H), 3.7-3.4 (br, m, 2H), 3.3-3.05 (br, m, 2H), 2.1-1.8 (br, m, 6H), 1.4-1.2 (m, 1H), 1.1-0.9 (m, 1H), 0.78 (t, J=7 Hz, 3H); ¹³C NMR δ 196.2, 137.4, 129.7, 126.3, 67.8, 51.9, 31.3, 22.9, 17.2, 13.7; APCI MS m/z 300 (M+1); Anal. (C₁₆H₂₁ClF₃NO) C, H, N, Cl.

### Example 17

Compound 0-2480 2-Pyrrolidin-1-yl-1-m-tolyl-pentan-1-one, hydrogen chloride salt. This compound was prepared, in 53% yield, as described in General Procedure A, with slight, modifications; Mp 166° C. (dec.); ¹H NMR δ 10.8-10.6 (br, 1H), 7.90 (d, 2H), 7.65-7.5 (m, 2H), 5.57 (m, 1H), 3.7-3.55 (br, 1H), 3.55-3.4 (br, 1H), 3.3-3.15 (br, m, 1H), 3.15-3.0 (br, m, 1H), 2.42 (s, 3H), 2.1-1.8 (br, m, 6H), 1.35-1.15 (m, 1H), 1.15-0.95 (m, 1H), 0.78 (t, J=7 Hz, 3H); ¹³C NMR δ 196.7, 138.8, 135.6, 134.5, 129.1, 126.1, 67.4, 53.6, 51.9, 31.7, 22.9, 20.8, 17.3, 13.7; APCI MS m/z 246 (M+1); Anal. (C₁₆H₂₄ClNO) C, H, N, Cl.

### Example 18

Compound 0-2479 2-Pyrrolidin-1-yl-1-o-tolyl-pentan-1-one, hydrogen chloride salt. This compound was prepared, in 39% yield, as described in General Procedure A, however, we were unable to obtain a crystalline sample of the compound. The hydrogen chloride salt was taken up in H₂O and lyophilized; ¹H NMR δ 10.9-10.7 (br, 1H), 8.12 (d, 1H), 7.58 (t, 1H), 7.44 (t, 2H), 5.56 (m, 1H), 3.7-3.5 (br, 2H), 3.35-3.1 (br, m, 2H), 2.46 (s, 3H), 2.1-1.7 (br, m, 6H), 1.4-1.2 (m, 1H), 1.1-0.9 (m, 1H), 0.76 (t, J=7 Hz, 3H); ¹³C NMR δ 199.1, 138.8, 134.4, 133.2, 132.3, 130.0, 126.2, 68.9, 53.5, 51.8, 31.4, 23.0, 20.7, 17.5, 13.7; APCI MS m/z 246 (M+1); Anal. (C₁₆H₂₄ClNO. 92/100H₂0) C, H, N, Cl.

### Example 19

Compound 0-2477 2-Pyrrolidin-1-yl-methyl-1 p-tolyl-pentan-1-one, hydrogen chloride salt. This compound was prepared from 1-o-Tolyl-pentan-1-one (3.5 g, 20 mmol) using the same method as described for General Procedure A with the following modifications. No chromatography was performed. The hydrogen chloride salt of the crude free base isolated after extraction of the crude reaction mixture into 1 M aqueous HCl and back-extraction (with 20% aqueous Na₂C0₃) into Et₂O in the usual way, was recrystallized from EtOH/Et₂O to give pure crystalline 2-pyrrolidin-1-yl-methyl-1 p-tolyl-pentan-1-one, as its hydrogen chloride salt (x) (2.6 g, 44%). Mp 176° C. (dec.); ¹H NMR δ 10.8-10.6 (br, 1H), 7.98 (d, 2H), 7.39 (d, 2H), 4.25-4.15 (br, m, 1H), 3.65-3.5 (m, 2H), 3.5-3.25 (m, 2H), 3.1-2.95 (br, m, 1H), 2.95-2.8 (br, m, 1H), 2.40 (s, 3H), 2.0-1.75 (m, 4H), 1.7-1.4 (m, 2H), 1.3-1.1 (m, 2H), 0.81 (t, J=7 Hz, 3H); ¹³C NMR δ 200.4, 144.4, 135.2, 129.7, 129.5, 128.7, 128.5, 54.0, 53.7, 53.3, 41.9, 33.5, 22.8, 22.3, 21.1, 19.0, 13.8; APCI MS m/z 260 (M+1); Anal. (C₁₇H₂₆CINO) C, H, N, Cl.

### Example 20

Compound 0-2478 1-(3,4-Dichloro-phenyl)-2-pyrrolidin-1-yl-methyl-pentan-1-one, hydrogen chloride salt. 2-Bromo-1-(3,4-dichloro-phenyl)-pentan-1-one (3.5 g, 15 mmol), pyrrolidine.HCl (2.4 g, 23 mmol) and paraformaldehyde (1.35 g, 45 mmol) were taken up in ⁱPrOH (25 mL) containing concentrated HCI (0.2 mL). The mixture was refluxed for 16 h. The solvent was removed by rotary evaporation and the residue was separated between 1 M aqueous HCI and Et₂O. The aqueous extracts were basified with 20% aqueous Na₂CO₃ to pH 8-9 and the organics were extracted into Et₂O. The organics were dried (MgSO₄), filtered, and reduced to an oil in vacuo. Column chromatography (10% McOH/CH₂Cl₂) gave the pure free base. The hydrogen chloride salt was prepared by reaction with 2 M ethereal HCI and filtration of the resulting white precipitate. Thus, 1-(3,4-Dichloro-phenyl)-2-pyrrolidin-1-yl-methyl-pentan-1-one, hydrogen chloride salt (0.61 g, 12%). Mp 168° C. (dec.); ¹H NMR δ 10.7-10.5 (br, 1H), 8.29 (d, 1H), 8.05 (dd, 1H), 7.88 (d, 1H), 4.3-4.1 (br, 1H), 3.7-3.5 (br, m, 2H), 3.5-3.25 (br, m, 2H), 3.15-2.85 (br, m, 2H), 2.1-1.75 (br, m, 4H), 1.75-1.4 (m, 2H), 1.35-1.05 (m, 2H), 0.81 (t, J=7 Hz, 3H); ¹³C NMR δ 198.9, 136.6, 135.9, 132.1, 131.4, 131.2, 130.5, 130.3, 128.7, 128.5, 54.1, 53.4, 42.3, 42.2, 33.1, 22.7, 22.4, 18.8, 13.8; APCI MS m/z 314, 312, 310 (M+1); Anal. (C₁₆H₂₂Cl₃NO) C, H, N, Cl.

### Example 21

Compound 0-2446 2-Pyrrolidin-1-yl-1-(4-N-methylpyrrole-phenyl)-pentan-1-one, hydrogen chloride salt. A cooled (−78° C.) solution of N-Methylpyrrole (1.14 g, 14 mmol) in THF (10 mL) was treated with ^{t}BuLi (9.1 mL of a 1.7M solution in pentane, 15 mmol) in a drop-wise fashion. The mixture was then warmed to room temperature for 2 h, then cooled to −78° C. Chlorotributylstannane (5.0 g, 15 mmol) was added to the mixture in a drop-wise fashion. On completion of addition, the mixture was warmed to room temperature and stirred for 1 h. The mixture was filtered and reduced to an oil in vacuo. This oil (crude 2-tributylstannyl-(N-methylpyrrole)) was added to a solution of 2-Pyrrolidin-1-yl-1-(4′-bromo-phenyl)-pentan-1-one (which had been freed from its hydrogen chloride salt by treatment with 20% aqueous .Na₂CO₃ and extraction into Et₂O) in dioxane (30 mL). The resulting solution was degassed by purging with N₂. [Pd(PPh₃)₄] (264 mg, 0.22 mmol) was added and the mixture was heated to 95-100° C. (oil bath temperature) for a period of 10 h. The solvent was removed in vacuo. The pure free base was obtained by column chromatography (5% McOH/CH₂Cl₂) as a yellow oil. The hydrogen chloride salt was prepared by treatment with 2M ethereal HCI. Lyophilization of an aqueous solution of the salt afforded a pale green solid characterized as 2-Pyrrolidin-1-yl-1-(4-N-methylpyrrole-phenyl)-pentan-1-one, as its hydrogen chloride salt (1.4 g, 36%). ¹H NMR δ 10.6-10.45 (br, IH), 8.11 (d, 2H), 7.72 (d, 2H), 7.00 (dd, 1H), 6.45 (dd, 1H), 6.15 (dd, 1H), 5.54 (m, 1H), 3.77 (s, 3H), 3.7-3.55 (br, 1H), 3.55-3.4 (br, 1H), 3.35-3.15 (br, m, IH), 3.15-3.0 (br, m, IH), 2.1-1.85 (br, m, 6H), 1.35-1.2 (m, 1H), 1.2-1.0 (m, 1H), 0.82 (t, J=7 Hz, 3H); ¹³C NMR δ 195.6, 139.1, 131.9, 131.5, 129.4, 127.4, 127.1, 111.1, 108.2, 67.2, 53.7, 51.9, 35.6, 31.9, 22.9, 17.4, 13.7; APCI MS m/z 311 (M+1); Anal. (C₂₀H₂₇ClN₂0.⅔H₂0) C, H, N, Cl.

### Example 22

Compound 0-2438 2-Pyrrolidin-1-yl-1-(4-thiophen-2-yl-phenyl)-pentan-1-one, hydrogen chloride salt. This compound was prepared using a procedure analogous to that described General Procedure A, except that commercially available 2-tributylstannyl thiophene was employed as a starting material, and chromatography was not performed on the crude free base. The crude hydrogen chloride salt was readily obtained by treatment of the crude free base with 2M ethereal HCI. Recrystallization from hot EtOH gave the title compound as a colorless crystalline solid (1.23 g, 61%). Mp 220° C. (dec.); ¹H NMR (DMSO-d6+12 drops CD₃0H) δ 8.12 (d, 2H), 7.93 (d, 2H), 7.77 (dd, 1 H), 7.72 (dd, 1 H), 7.23 (dd, 1 H), 5.5-5.4 (br, 1 H), 3.7-3.45 (br, m, 2H), 3.3-3.2 (br, m, 1H), 3.1-3.0 (br, m, 1H), 2.2-1.9 (br, m, 6H), 1.35-1.2 (m, 1H), 1.2-1.0 (m, IH), 0.83 (t, J=7 Hz, 3H); ¹³C NMR δ 195.9, 141.8, 140.3, 132.9, 130.3, 129.3, 128.6, 126.6, 126.0, 68.1, 54.5, 52.1, 32.2, 23.1, 17.4, 13.8; APCI MS m/z 314 (M+1); Anal. (C₁₉H₂₄ClNOS) C, H, N, Cl.

### Example 23

Compound 0-2441 2-Pyrrolidin-1-yl-1-furan-2-yl-phenyl)-pentan-1-one, hydrogen chloride salt. This compound was prepared using a procedure analogous to that previously described except that commercially available 2-tributylstannyl furan was employed as a starting material, and chromatography was not performed on the crude free base. The crude hydrogen chloride salt was recrystallized from hot EtOH to give pure (1.13 g, 59%) as a colorless crystalline solid Mp 236° C. (dec.); ¹H NMR (DMSO-d6+6 drops CD₃OH) δ 8.14 (d, 2H), 7.95 (d, 2H), 7.90 (d, 1 H), 7.29 (d, 1 H), 6.71 (dd, 1 H), 5.51 (m, 1 H), 3.7-3.6 (br, m, 1 H), 3.6-3.45 (br, m, 1 H), 3.35-3.2 (br, m, 1 H), 3.15-3.0 (br, m, 1 H), 2.15-1.85 (br, m, 6H), 1.35-1.15 (m, 1 H), 1.15-1.0 (m, 1H), 0.81 (t, J=7 Hz, 3H); ¹³C NMR δ 195.7, 151.8, 145.1, 136.0, 132.6, 130.0, 123.8, 112.9, 109.9, 67.8, 54.2, 52.0, 32.0, 22.9, 17.3, 13.7; APCI MS m/z 298 (M+1); Anal. (C₁₉H₂₄CIN0₂) C, H, N, Cl.

### Example 24

Compound 0-2443 2-Pyrrolidin-1-yl-1-(4-nitro-phenyl)-pentan-1-one, hydrogen chloride salt. A 50% w/w aqueous solution of H₂0₂ (7 mL, 0.12 mol) was added to CH₂Cl₂, (50 mL which had been cooled on an ice bath. Trifluoroacetic anhydride (23 mL, 0.14 mol) was added slowly via syringe, then the solution was warmed to room temperature. N-[4-(2-Pyrrolidin-1-yl-pentanoyl)-phenyl]-acetamide, hydrogen chloride salt (4.5 g, 18 mmol) was added over 20 min, then the mixture was heated to reflux for 1 h. The solution was cooled, then quenched cautiously with aqueous Na₂SO₃ (100 mL of a 1.6 M solution, 0.16 mol). The organics were separated and extracted into Et₂O, then back-extracted into 1 M aqueous HCI. The acidic extracts were basified with 20% aqueous Na₂CO₃ to pH 8-9 and extracted into Et₂O. The organic extracts were dried (MgSO₄), filtered, then treated with 2 M ethereal HCI. The resulting white precipitate was collected on a frit, dissolved in water, then lyophilized to give pure 2-Pyrrolidin-1-yl-1-(4-nitro-phenyl)-pentan-1-one, as its hydrogen chloride salt (x) (290 mg, 5%). Mp 189° C. (dec.); ¹H NMR δ 10.8-10.6 (br, 1H), 8.45 (d, 2H), 8.32 (d, 2H), 5.65 (m, 1H), 3.7-3.3 (br, m, 2H), 3.3-3.1 (br, m, 2H), 2.1-1.8 (br, m, 6H), 1.4-1.2 (m, 1H), 1.1-0.9 (m, 1H), 0.78 (t, J=7 Hz, 3H); ¹³C NMR δ 196.0, 150.8, 138.7, 130.4, 124.3, 68.1, 53.9, 52.0, 31.2, 22.9, 17.2, 13.7; APCI MS m/z 277 (M+1); Anal. (C₁₅H₂₁ClN₂0₃. 42/100H₂0. 8/100HCI) C, H, N, Cl.

### Example 25

Compound 0-2439 N-[4-(2-Pyrrolidin-1-yl-pentanoyl)-phenyl]-acetamide, hydrogen chloride salt. This compound was prepared, in 56% yield, as described in General Procedure A, with slight modifications; Mp 195° C. (dec.); ¹H NMR δ 10.76 (s, 1H), 10.55-10.35 (br, 1H), 8.05 (d, 2H), 7.85 (d, 2H), 5.5-5.4 (br, m, 1H), 3.7-3.55 (br, 1H), 3.5-3.3 (br, 1H), 3.3-3.15 (br, m, 1H), 3.15-3.0 (br, m, 1H), 2.13 (s, 3H), 2.1-1.8 (br m, 6H), 1.3-1.15 (m, 1H), 1.15-1.0 (m, 1H), 0.79 (t, J=7 Hz, 3H); ¹³C NMR δ 194.8, 169.4, 145.4, 130.4, 128.8, 118.4, 67.0, 53.6, 51.9, 32.0, 24.2, 22.8, 17.4, 13.7; APCI MS m/z 289 (M+1); Anal. (C₁₇H₂₅CIN₂0₂.½H₂0) C, H, N, Cl.

### Example 26

Compound 0-2419 2-Pyrrolidin-1-yl-1-(4′-bromo-phenyl)-pentan-1-one, hydrogen chloride salt. This compound was prepared, in 62% yield, as described in General Procedure A, with slight modifications; Mp 200° C. (dec.); ¹H NMR δ 10.7-10.5 (br, 1 H), 8.03 (d, 2H), 7.87 (d, 2H), 5.56 (m, 1 H), 3.7-3.55 (br, m, 1 H), 3.55-3.4 (br, m, 1 H), 3.35-3.1 (br, m, 1 H), 3.1-3.0 (br, m, 1H), 2.1-1.8 (br, m, 6H), 1.4-1.2 (m, 1H), 1.15-0.95 (m, 1H), 0.78 (t, J=7 Hz, 3H); ¹³C NMR δ 196.0, 133.4, 132.4, 130.8, 129.4, 67.4, 53.7, 51.9, 31.6, 22.9, 17.3, 13.7; APCI MS m/z 312, 310 (M+1); Anal. (C₁₅H₂₁BrClNO) C, H, N, Cl.

### Example 27

Compound O-2418 2-Pyrrolidin-1-yl-1-(4′-hydroxy-phenyl)-pentan-1-one, hydrogen chloride salt. 2-Pyrrolidin-1-yl-1-(4′methoxy-phenyl)-pentan-1-one (9.00 g, 30.3 mmol) was freed from its hydrogen chloride salt by basification to pH 8-9 with 20% aqueous Na₂CO₃ and extraction into CH₂Cl₂. The free base was dissolved in CH₂Cl₂ (50 mL) and cooled to −78° C., whereon BBr₃ (90 mL, 1.0 M solution in CH₂Cl₂, 90 mmol) was added to the solution over 0.5 h. The mixture was stirred for a further 1 h before warming gradually to room temperature. The gummy mixture, which became difficult to stir was quenched after 2 h with saturated aqueous NaHCO₃ and the neutral organics were extracted into CH₂Cl₂. A white solid precipitated from the aqueous layer which was collected on a frit (1.8 g). Work-up of the organic layer in the usual way afforded a further 1 g of crude free base which was converted to its hydrogen chloride salt by reaction with 2 M ethereal HCl. The two solids were combined and recrystallized from hot ethanol to give pure 2-Pyrrolidin-1-yl-1-(4′-hydroxy-phenyl)-pentan-1-one, as its hydrogen chloride salt (2.9 g, 34%). Mp 235° C. (dec.); ¹H NMR (CD₃OD) δ 7.99 (d, 2H), 6.93 (d, 2H), 5.26 (t, J=5.5 Hz, 1H), 5.0-1.8 (s, br, 2H), 3.7-3.0 (br, 4H), 2.2-1.9 (br, m, 6H), 1.4-1.1 (m, 2H), 0.89 (t, J=7 Hz, 3H); ¹³C NMR δ 195.0, 156.8, 132.9, 127.3, 117.0, 69.8, 33.9, 24.1, 18.6, 14.2; APCI MS m/z 248 (M+1); Anal. (C₁₅H₂₂ClNO₂) C, H, N, Cl.

### Example 28

Compound O-2417 2-Pyrrolidin-1-yl-1-(4′-methoxy-phenyl)-pentan-1-one, hydrogen chloride salt. This compound was prepared 68% yield, as described in General Procedure A, with slight modifications; ¹H NMR δ 10.8-10.6 (br, 1H), 8.10 (d, 2H), 7.15 (d, 2H), 5.55 (m, 1H), 3.89 (s, 3H), 3.7-3.55 (br, m, 1H), 3.55-3.4 (br, m, 1H), 3.3-3.15 (br, m, 1H), 3.1-2.95 (br, m, 1H), 2.15-1.85 (br, m, 6H), 1.34-1.15 (m, 1H), 1.15-1.0 (m, 1H), 0.79 (t, J=7 Hz, 3H); ¹³C NMR δ 194.7, 164.5, 131.4, 127.4, 114.5, 66.7, 55.8, 53.4, 51.8, 32.0, 22.9, 17.5, 13.7; APCI MS m/z 262 (M+1); Anal. (C₁₆H₂₄ClNO₂.½H₂O.½HCl) C, H, N, Cl.

### Example 29

Compound O-2525 3-Pyrrolidin-1-yl-1-p-tolyl-pentan-1-one, hydrogen chloride salt. This compound was prepared from 1-p-Tolyl-pent-2-en-1-one using the procedure of General Procedure A). Mp 97° C. (dec.); ¹H NMR δ 11.1-10.9 (br, 1H), 7.94 (d, 2H), 7.38 (d, 2H), 3.9-3.75 (br, 1H), 3.7-3.6 (m, 1H), 3.6-3.3 (m, 3H), 3.15-2.95 (br, m, 2H), 1.96 (s, 3H), 2.0-1.8 (br, m, 5H), 1.8-1.6 (m, 1H), 0.88 (t, J=7 Hz, 3H); ¹³C NMR δ 196.2, 144.3, 133.5, 129.3, 128.3, 59.7, 50.7, 50.4, 37.9, 23.8, 22.9, 22.8, 21.2, 9.9; APCI MS m/z 246 (M+1); Anal. (C₁₆H₂₄ClNO) C, H, N, Cl.

### Example 30

Compound O-2524 1-(3,4-Dichloro-phenyl)-3-pyrrolidin-1-yl-pentan-1-one, hydrogen chloride salt. 1-(3,4-Dichloro-phenyl)-pen-2-en-1-one (1.29 g, 5.63 mmol) was taken up in EtOH (10 mL), cooled on an ice bath, and degassed by purging with N₂. Pyrrolidine (0.80 g, 11 mmol) was added dropwise over 2 min. After 0.5 h, the ethanolic solution was separated between 1M aqueous HCl and Et₂O. The HCl extracts were collected and back-extracted into Et₂0 by treatment with 20% aqueous Na₂CO₃. The ethereal extracts were dried (MgSO₄), filtered, and treated with 2M ethereal HCl. Laborious trituration afforded a white powder which was collected on a frit and washed copiously with Et₂O. This white powder was identified as 1-(3,4-Dichloro-phenyl)-2-pyrrolidin-1-yl-methyl-pentan-1-one, hydrogen chloride salt (0.99 g, 50%). Mp 104-107° C. (dec.); ¹HNMR δ 11.1-10.9 (br, 1H), 8.27 (d, 1H), 7.98 (dd, 1H), 7.87 (d, 1H), 3.9-3.35 (br, m, 5H), 3.15-2.95 (br, 2H), 2.05-1.8 (br, m, 5H), 1.8-1.6 (m, 1H), 0.90 (t, J=7 Hz, 3H); ¹³C NMR δ 195.0, 136.4, 136.1, 131.8, 131.1, 130.3, 128.1, 59.2, 50.7, 50.1, 38.2, 23.8, 22.9, 10.0; APCI MS m/z 300, 302, 304 (M+1); Anal. (C₁₅H₂₀Cl₃NO.⅓H₂O) C, H, N, Cl.

### Example 31

Compound O-2495 1-(3-Iodo-phenyl)-2-pyrrolidin-1-yl-pentan-1-one, hydrogen chloride salt. This compound was prepared, in 20% yield, as described in General Procedure A, with slight modifications; Mp 203° C. (dec.); ¹H NMR δ 10.6-10.4 (br, 1H), 8.39 (s, 1H), 8.14 (d, 1H), 8.07 (d, 1H), 7.44 (t, 1H), 5.51 (m, 1H), 3.7-3.55 (br, m, 1H), 3.55-3.4 (br, m, 1H), 3.3-3.15 (br, m, 1H), 3.15-3.0 (br, m, 1H), 2.1-1.8 (br, m, 6H), 1.35-1.15 (m, 1H), 1.1-0.9 (m, 1H), 0.79 (t, J=7 Hz, 3H); ¹³C NMR δ 195.7, 143.3, 136.9, 136.1, 131.8, 131.3, 128.0, 95.7, 67.5, 53.8, 51.9, 31.5, 22.8, 17.2, 13.6; APCI MS m/z 358 (M+1); Anal. (C₁₅H₂₁ClINO) C, H, N, Cl.

### Example 32

Compound O-2390 2-Pyrrolidin-1-yl-1-(3,4-Dichloro-phenyl)-pentan-1-one, hydrogen chloride salt. This compound was prepared, in 32% yield, as described in General Procedure A, with slight modifications; Mp 195° C. (dec.); ¹H NMR δ 10.8-10.6 (br, 1H), 8.35 (d, 1H), 8.04 (dd, 1H), 7.94 (d, 1H), 5.58 (m, 1H), 3.7-3.6 (br, 1H), 3.6-3.45 (br, m, 1H), 3.3-3.05 (br,m, 2H), 2.15-2.85 (br, m, 6H), 1.35-1.15 (m, 1H), 1.15-0.95 (m, 1H), 0.79 (t, J=7 Hz, 3H); ¹³C NMR δ 195.0, 137.8, 134.5, 132.3, 131.6, 130.8, 128.8, 67.5, 53.7, 51.9, 31.4, 22.9, 17.2, 13.6; APCI MS m/z 300, 302, 304 (M+1); Anal. (C₁₅H₂₀Cl₃NO) C, H, N, Cl.

### Example 33

Compound O-2389 2-Butylamin-1-yl-1-(3,4-dichloro-phenyl)-pentan-1-one, hydrogen chloride salt. This compound was prepared, in 69% yield, as described in General Procedure A, with slight modifications; Mp 185° C. (dec.); ¹H NMR δ 9.8-9.6 (br, 1H), 9.3-9.1 (br, 1H), 8.35 (d, 1H), 8.04 (dd, 1H), 7.91 (d, 1H), 5.4-5.25 (br, 1H), 3.05-2.75 (br, m, 2H), 2.05-1.8 (br, m, 2H), 1.8-1.6 (br, m, 2H), 1.4-1.2 (m, 3H), 1.2-1.0 (m, 1H), 0.88 (t, J=7 Hz, 3H), 0.78 (t, J=7 Hz, 3H); ¹³C NMR δ 194.8, 137.6, 134.3, 132.3, 131.5, 130.6, 128.7, 60.8, 45.7, 31.5, 27.4, 19.3, 17.2, 13.6, 13.5; APCI MS m/z 302, 304, 306 (M+1); Anal. (C₁₅H₂₂Cl₃NO) C, H, N, Cl.

### Example 34

Compound O-2388 2-Piperidin-1-yl-1-(3,4-dichloro-phenyl)-pentan-1-one, hydrogen chloride salt. This compound was prepared, in 35% yield, as described in General Procedure A, with slight modifications; Mp 202° C. (dec.); ¹H NMR δ 10.5-10.3 (br, 1H), 8.40 (d, 1H), 8.10 (dd, 1H), 7.94 (d, 1H), 5.45-5.35 (br, m, 1H), 3.7-3.55 (br, m, 1H), 3.45-3.3 (br, m, 1H), 3.2-1.95 (br, m, 2H), 2.1-1.65 (br, m, 7H), 1.5-1.3 (br, 1H), 1.2-1.0 (br, m, 2H), 0.81 (t, J=7 Hz, 3H); ¹³C NMR δ 195.3, 138.0, 135.3, 132.4, 131.6, 130.7, 128.8, 65.8, 52.0, 50.2, 29.3, 22.3, 22.0, 21.5, 17.8, 13.7; APCI MS m/z 314, 316, 318 (M+1); Anal. (C₁₆H₂₂Cl₃NO) C, H, N, Cl.

### Example 35

Compound O-2387 2-Pyrrolidin-1-yl-phenyl-pentan-1-one, hydrogen chloride salt. This compound was prepared, in 50% yield, as described in General Procedure A, with slight modifications; Mp 173° C. (dec.); ¹H NMR δ 10.85-10.65 (br, 1H), 8.11 (d, 2H), 7.78 (t, 1H), 7.64 (t, 2H), 5.62 (m, 1H), 3.7-3.55 (br, 1H), 3.55-3.4 (br, m, 1H), 3.35-3.2 (br, m, 1H), 3.15-3.0 (br, m, 1H), 2.15-1.85 (br, m, 6H), 1.4-1.2 (m, 1H), 1.15-0.95 (m, 1H), 0.78 (t, J=7 Hz, 3H); ¹³C NMR δ 196.7, 134.9, 134.5, 129.2, 128.8, 67.3, 53.6, 51.9, 31.7, 22.9, 17.4, 13.7; APCI MS m/z 232 (M+1); Anal. (C₁₅H₂₂ClNO) C, H, N, Cl.

### Example 36

Compound O-2384 2-Pyrrolidin-1-yl-1-(3,4-dichloro-phenyl)butan-1-one, hydrogen chloride salt. This compound was prepared, in 71% yield, as described in General Procedure A, with slight modifications; Mp 211° C. (dec.); ¹H NMR δ 10.95-10.75 (br, 1H), 8.35 (d, 1H), 8.06 (dd, 1H), 7.92 (d, 1H), 5.75-5.65 (br, m, 1H), 3.65-3.35 (br, m, 2H), 3.3-3.1 (br, m, 1H), 2.15-1.9 (br, m, 6H)), 0.78 (t, J=7 Hz, 3H); ¹³C NMR δ 194.7, 137.7, 134.5, 132.3, 131.6, 130.7, 128.8, 68.5, 53.7, 51.8, 23.0, 22.6, 8.4; APCI MS m/z 286, 288, 290 (M+1); Anal. (C₁₄H₁₈Cl₃NO) C, H, N.

### Example 37

Compound O-2370 2-Pyrrolidin-1-yl-1-(4′-fluoro-phenyl)-pentan-1-one, hydrogen chloride salt. This compound was prepared, in 78% yield, as described in General Procedure A, with slight modifications; Mp 218° C. (dec.); ¹H NMR δ 10.7-10.5 (br, 1H), 8.19 (m, 2H), 7.49 (t, 2H), 5.6-5.5 (br, m, 111), 3.7-3.55 (br, 1H), 3.55-3.4 (br, 1H), 3.3-3.15 (br, m, 1H), 3.15-3.0 (br, 1H), 2.15-1.8 (br, m, 6H), 1.35-1.15 (m, 1H), 1.15-0.95 (m, 1H), 0.79 (t, J=7 Hz, 3H); ¹³C NMR δ 195.2, 132.2, 132.0, 131.3, 116.6, 116.3, 67.2, 53.5, 51.9, 31.7, 22.9, 17.4, 13.7; APCI MS m/z 250 (M+1); Anal. (C₁₅H₂₁ClFNO) C, H, N, Cl.

### Example 38

Compound O-2371 2-Pyrrolidin-1-yl-1-p-tolyl-pentan-1-one, hydrogen chloride salt. This compound was prepared, in 68% yield, as described in General Procedure A, with slight modifications; Mp 180° C. (dec.); ¹H NMR δ 10.8-10.65 (br, 1H), 8.01 (d, 2H), 7.44 (d, 2H), 5.56 (m, 1H), 3.7-3.55 (br, 1H), 3.55-3.4 (br, m, 1H), 3.35-3.2 (br, m, 1H), 3.15-3.0 (br, m, 1H), 2.42 (s, 3H), 2.15-1.85 (br, m, 6H), 1.4-1.2 (m, 1H), 1.15-0.95 (m, 1H), 0.78 (t, J=7 Hz, 3H); ¹³C NMR δ 196.1, 145.8, 132.1, 129.8, 129.0, 67.1, 53.5, 51.9, 31.8, 22.9, 21.3, 17.4, 13.7; APCI MS m/z 246 (M+1); Anal. (C₁₆H₂₄ClNO.⅙H₂O) C, H, N, Cl.

### Example 39

Compound O-2440 and Compound O-2442 (2R)-2-Pyrrolidin-1-yl-1-p-tolyl-pentan-1-one, hydrogen chloride salt (O-2440) and (2S)-2-Pyrrolidin-1-yl-1-p-tolyl-pentan-1-one, hydrogen chloride salt (O-2442). Pyrovalerone.HCl (10.0 g, 35.5 mmol) was freed from its hydrogen chloride salt by extraction into Et₂O from 20% aqueous Na₂CO₃ at pH 8-9. The free base was dissolved in EtOH (50 mL) and heated until nearly boiling. Dibenzoyl-D-tartaric acid (12.7 g, 35.5 mmol) in hot ethanol (150 mL) was added all at once to the pale yellow solution of free base. The resulting colorless solution was refluxed for 1 min, cooled, and the solvent was removed in vacuo. The residue was dissolved in CH₂Cl₂ (530 mL) and hexanes (700 mL) were added with swirling. After 3 d, the resulting crystalline solid (9.1 g) was-collected on a frit. Analysis by ¹H NMR in CDCl₃ showed that this material had a diastereomeric excess (d.e.) of 70-75%. A further three recrystallizations from CH₂Cl₂/hexanes (300 mL/400 mL) gave a single diastereoisomer (6.1 g, 61%). Mp 100-120° C.; ¹H NMR δ 8.10 (d, 4H), 7.87 (d, 2H), 7.51 (t, 2H), 7.37 (t, 2H), 7.18 (d, 2H), 5.91 (s, 2H), 5.37 (t, 1H), 3.75 (br, m, 2H), 2.32 (s, 3H), 2.0-1.8 (br, m, 6H), 1.4-1.1 (br, m, 4H), 0.71 (t, 3H). XRD analysis of this compound showed it to be a salt of dibenzoyl-D-tartaric acid and (1R)-2-Pyrrolidin-1-yl-1-p-tolyl-pentan-1-one. The dibenzoyltartarate salt was dissolved in 20% aqueous Na₂CO₃ and extracted into Et₂O. The Et₂O layer was collected, dried and filtered. The hydrogen chloride salt was prepared by adding 2 M ethereal HCl to this solution. The resulting white solid was recrystallized from EtOH/Et₂O to give pure (1R)-2-Pyrrolidin-1-yl-1-p-tolyl-pentan-1-one as its hydrogen chloride salt. The physical properties of this compound are identical with those of the racemic material.

The residues from recrystallization of the dibenzoyl-D-tartaric acid-(1R)-2-Pyrrolidin-1-yl-1-p-tolyl-pentan-1-one were combined and the free base was liberated by reaction with 20% aqueous Na₂CO₃. The ethereal extracts were washed once with 20% aqueous Na₂CO₃, dried (MgSO₄), filtered, and reduced to an oil (5.2 g, 21 mmol) in vacuo. This oil was taken up in hot EtOH (50 mL), and a solution of dibenzoyl-1-tartaric acid (7.5 g, 21 mmol) in hot EtOH (100 mL) was added with swirling. The mixture was refluxed for 1 min, cooled, then the solvent was removed in vacuo. Four recrystallizations, as described above, gave a single diastereoisomer (5.4 g, 50%). XRD analysis showed that this material was a diastereomeric salt of dibenzoyl-1-tartaric acid-(1S)-2-Pyrrolidin-1-yl-1-p-tolyl-pentan-1-one. The hydrogen chloride salt was prepared as described above for (1R)-2-Pyrrolidin-1-yl-1-p-tolyl-pentan-1-one.

Compounds can be prepared by α-bromination of analogous ketones by the following general procedure:

General Procedure B. The ketone (as a solution in Et₂O, or CH₂Cl₂ (for less soluble substrates)) was cooled on an ice bath and anhydrous AlCl₃ was added to the solution (catalytic quantity, 1-5 mol %). Bromine (approximately 0.1 mol eq) was added to the solution all at once. Typically, after 10 min the solution changed from a light orange to colorless (if this change did not occur at 0° C., then the flask was warmed to room temperature). The remaining bromine (0.9 mol eq) was then added to the solution in a drop-wise manner over 5 min. The solution was neutralized (aqueous NaHCO₃), separated, dried (MgSO₄), filtered, and reduced to a lightly colored oil in vacuo. Yields were quantitative and the crude materials were judged to be sufficiently pure by ¹H NMR for use directly in the subsequent step.

### Example 40

4-(2-Bromo-pentanoyl)-benzonitrile. ¹H NMR δ 8.11 (d, 2H), 7.80 (d, 2H), 5.07 (dd, 1H), 2.25-2.05 (m, 2H), 1.7-1.35 (m, 2H), 1.00 (t, 3H).

### Example 41

2-Bromo-(3,4-dimethoxy-phenyl)-pentan-1-one, and 2-Bromo-1-(2-bromo-4,5-dimethoxy-phenyl)-pentan-1-one. These two compounds were produced together by General Procedure B and were separated by careful chromatography (10% EtOAc/hexanes). 2-Bromo-1-(3,4-dimethoxy-phenyl)-pentan-1-one; ¹H NMR δ 7.66 (dd, 1H), 7.58 (d, 1H), 6.91 (d, 1H), 5.15 (dd, 1H), 3.97 (s, 3H), 3.95 (s, 3H), 2.25-2.05 (m, 2H), 1.7-1.35 (m, 2H), 1.01 (t, 3H). 2-Bromo-1-(2-bromo-4,5-dimethoxy-phenyl)-pentan-1-one; ¹H NMR δ 7.07 (s, 1H), 7.04 (s, 1H), 5.28 (dd, 1H), 3.92 (s, 3H), 3.90 (s, 3H), 2.3-2.0 (m, 2H), 1.7-1.4 (m, 2H), 1.00 (t, 3H).

### Example 42

2-Bromo-4-methyl-1-p-tolyl-pentan-1-one. ¹H NMR δ 7.92 (d, 2H), 7.29 (d, 2H), 5.21 (dd, 1H), 2.43 (s, 3H), 2.15-1.95 (m, 2H), 1.95-1.75 (m, 1H), 0.96 (d, 6H).

### Example 43

2-Bromo-1-(4-iodo-phenyl)-pentan-1-one. ¹H NMR δ 7.85 (d, 2H), 7.72 (d, 2H), 5.06 (dd, 1H), 2.25-2.05 (m, 2H), 1.65-1.35 (m, 2H), 0.98 (t, 3H).

### Example 44

2-Bromo-1-(4-trifluoromethyl-phenyl)-pentan-1-one. ¹H NMR δ 8.13 (d, 2H), 7.76 (d, 2H), 5.11 (dd, 1H), 2.25-2.1 (m, 2H), 1.7-1.4 (m, 2H), 1.00 (t, 3H).

### Example 45

2-Bromo-1-naphthalen-2-yl-pentan-1-one. ¹H NMR δ 8.55 (s, 1H), 8.1-7.85 (m, 4H), 7.60 (m, 2H), 5.33 (dd, 1H), 2.3-2.1 (m, 2H), 1.7-1.4 (m, 2H), 1.01 (t, 3H).

### Example 46

2-Bromo-1-o-tolyl-pentan-1-one. 7.63 (d, 1H), 7.42 (m, 1H), 7.27 (m, 2H), 5.05 (dd, 1H), 2.25-2.0 (m, 2H), 1.65-1.35 (m, 2H), 0.99 (t, 3H).

### Example 47

2-Bromo-1-(4-bromo-phenyl)-pentan-1-one. ¹H NMR δ 7.88 (d, 2H), 7.63 (d, 2H), 5.06 (dd, 1H), 2.25-2.05 (m, 2H), 1.65-1.35 (m, 2H), 0.99 (t, 3H).

### Example 48

N-[4-(2-Bromo-pentanoyl)-phenyl]-acetamide. ¹H NMR δ 8.00 (d, 2H), 7.65 (br, m, 3H), 5.12 (dd, 1H), 2.23 (s, 3H), 2.2-2.05 (m, 2H), 1.7-1.35 (m, 2H), 0.98 (t, 3H).

### Example 49

4-(2-Bromo-pentanoyl)-benzoic acid methyl ester. ¹H NMR δ 8.14 (d, 2H), 8.06 (d, 2H), 5.13 (t, 1H), 3.96 (s, 3H), 2.2-2.05 (m, 2H), 1.65-1.35 (m, 2H), 1.00 (t, 3H).

### Example 50

2-Bromo-1-(4-hydroxymethyl-phenyl)-pentan-1-one. ¹H NMR δ 8.01 (d, 2H), 7.48 (d, 2H), 5.15 (dd, 1H), 4.79 (br, d, 2H), 2.25-2.05 (m, 2H), 2.05-1.95 (br, 1H), 1.65-1.4 (m, 2H), 0.99 (t, 3H).

### Example 51

2-Bromo-1-(4-fluoro-phenyl)-pentan-1-one. ¹H NMR δ 8.05 (dd, 2H), 7.16 (dd, 2H), 5.09 (dd, 1H), 2.25-2.05 (m, 2H), 1.7-1.35 (m, 2H), 0.99 (t, 3H).

### Example 52

2-Bromo-1-phenyl-pentan-1-one. ¹H NMR δ 8.02 (d, 2H), 7.62 (m, 1H), 7.49 (t, 2H), 5.15 (dd, 1H), 2.25-2.05 (m, 2H), 1.7-1.4 (m, 2H), 0.99 (t, 3H).

### Example 53

2-Bromo-1-(3,4-dichloro-phenyl)-butan-1-one. ¹H NMR δ 8.09 (d, 1H), 7.84 (dd, 1H), 7.57 (d, 1H), 4.95 (dd, 1H), 2.35-2.05 (m, 2H), 1.09 (t, 3H).

### Example 54

2-Bromo-1-(3,4-dichloro-phenyl)-pentan-1-one. ¹H NMR δ 8.09 (d, 1H), 7.84 (dd, 1H), 7.55 (d, 1H), 5.02 (dd, 1H), 2.25-2.05 (m, 2H), 1.65-1.35 (m, 2H), 0.99 (t, 3H).

### Example 55

2-Bromo-1-p-tolyl-pentan-1-one. ¹H NMR δ 7.92 (d, 2H), 7.29 (d, 2H), 5.14 (dd, 1H), 2.43 (s, 3H), 2.25-2.05 (m, 2H), 1.65-1.35 (m, 2H), 0.98 (t, 3H)

### Example 56

2-Bromo-1-(4-methoxy-phenyl)-pentan-1-one. ¹H NMR δ 8.01 (d, 2H), 6.96 (d, 2H), 5.12 (dd, 1H), 3.89 (s, 3H), 2.25-2.05 (m, 2H), 1.65-1.35 (m, 2H), 0.98 (t, 3H).

The ketones were prepared (except where noted) by alkylation of the analogous commercially available nitrile compounds, followed by acidic hydrolysis by the following method:

General Procedure C. The nitrile (10 mmol) was added in several portions, over 0.5 h to a solution of the ⁿBuMgCl (12 mmol) in toluene (20 mL). The reactions were monitored by TLC and heated where necessary. Generally, after 2 h stirring at room temperature, the reaction was complete. The reaction mixture was poured onto ice and concentrated H₂SO₄ (2 mL) was added. Hydrolysis of the intermediate imine usually occurred at room temperature after several minutes, however, for some substrates, heating was necessary to effect this transformation. The organics were extracted into Et₂O, dried (MgSO₄), filtered, and reduced to an oil in vacuo.

### Example 57

N-(4-Pentanoyl-phenyl)-acetamide. Acetanilide (15.0 g, 111 mmol) was taken up in CS₂ and valeryl chloride (22.5 g, 186 mmol) was added in one portion. AICl₃ (44 g, 330 mmol) was added in 2 g portions to the resulting solution over a period of 0.5 h. The translucent mixture was heated to reflux for 18 h. On cooling, the top layer of CS₂ was decanted from the remaining brown oil which was subsequently poured onto ice containing concentrated HCl (10 mL). The resulting gummy orange solid was collected by filtration, washed with saturated aqueous NaHCO₃, then a small volume of Et₂O and dried in air. Recrystallization from hot MeOH gave pure N-(4-Pentanoyl-phenyl)-acetamide (14.7 g, 60%) as a colorless solid. ¹H NMR δ 7.94 (d, 2H), 7.61 (d, 2H), 7.41 (br, s, 1H), 2.94 (t, 2H), 2.22 (s, 3H), 1.8-1.65 (m, 2H), 1.45-1.35 (m, 2H), 0.95 (t, 3H); ¹³C NMR δ 168.4, 142.0, 132.9, 129.5, 118.8, 38.2, 26.6, 24.8, 22.5, 14.0.

### Example 58

1-(3,4-Dichloro-phenyl)-pentan-1-one. Following General Procedure C, this compound was prepared in 93% yield and employed in the next step of the reaction as the crude material. ¹H NMR δ 8.03 (d, 1H), 7.78 (dd, 1H), 7.54 (d, 1H), 2.92 (t, 2H), 1.71 (m, 2H), 1.39 (m, 2H), 0.94 (t, 3H).

### Example 59

1-(3,4-Dichloro-phenyl)-butan-1-one. Following General Procedure C, this compound was prepared in 100% yield and employed in the next step of the reaction as the crude material ¹H NMR δ 8.01 (d, 1H), 7.78 (dd, 1H), 7.54 (d, 1H), 2.91 (t, 2H), 1.77 (sextet, 2H), 1.01 (t, 3H).

### Example 60

1-(3,4-Dimethoxy-phenyl)-pentan-1-one. This compound was prepared following General Procedure C. The crude material was further purified by distillation (Bp 131° C., 0.05 mmHg) to give the pure title compound in 80% yield. ¹H NMR δ 7.60 (dd, 1H), 7.54 (d, 1H), 6.89 (d, 1H), 3.95 (s, 3H), 3.94 (s, 3H), 2.93 (t, 2H), 1.72 (m, 2H), 1.42 (m, 2H), 0.96 (t, 3H).

### Example 61

4-Methyl-1-p-tolyl-pentan-1-one. This compound was prepared in quantitative yield by Friedel Crafts acylation of toluene with valeryl chloride. ¹H NMR δ 7.86 (d, 2H), 7.26 (d, 2H), 3.94 (t, 2H), 2.41 (s, 3H), 1.62 (m, 3H), 0.94 (d, 6H).

### Example 62

1-(4-Trifluoromethyl-phenyl)-pentan-1-one. Following General Procedure C, this compound was prepared in 95% yield and employed in the next step of the reaction as the crude material. ¹H NMR δ 8.06 (d, 2H), 7.43 (d, 2H), 3.00 (t, 2H), 1.74 (m, 2H), 1.41 (m, 2H), 0.96 (t, 3H).

### Example 63

1-Naphthalen-2-yl-pentan-1-one. Following General Procedure C, this compound was prepared in 95% yield and employed in the next step of the reaction as the crude material. ¹H NMR δ 8.48 (s, 1H), 8.04 (dd, 1H), 7.97 (d, 1H), 7.90 (m, 2H), 7.57 (m, 2H), 3.11 (t, 2H), 1.79 (m, 2H), 1.44 (m, 2H), 0.98 (t, 3H).

### Example 64

1-(3,4-Dichloro-phenyl)-pen-2-en-1-one. 2-Bromo-1-(3,4-dichloro-phenyl)-pentan-1-one (3.36 g, 10.9 mmol) was dissolved in DMF (60 mL). Li₂CO₃ (1.28 g, 17 mmol) and LiBr (0.99 g, 11.5 mmol) was added to the solution which was then heated with stirring to 110-120° C. (oil bath temperature) for 1.5 h. The mixture was diluted with H₂O (100 mL) and the organics were extracted into EtOAc (3×50 mL). The ethyl acetate layer was collected and washed with saturated brine (2×50 mL), dried (MgSO₄), filtered, and reduced to an oil in vacuo. Careful column chromatography (1% EtOAc/hexanes-2.5% EtOAc/hexanes) furnished the pure compound as a colorless solid (1.5 g, 60%). ¹H NMR δ 8.01 (d, 1H), 7.76 (dd, 1H), 7.55 (d, 1H), 7.15 (dt, 1H), 6.80 (dt, 1H), 2.37 (m, 2H), 1.15 (t, 3H); ¹³C NMR δ 188.5, 152.8, 137.6, 137.1, 133.2, 130.6, 130.5, 127.5, 124.1, 26.0, 12.2.

### Example 65

1-p-Tolyl-pent-2-en-1-one. This compound was prepared as described for General Procedure C employing 2-Bromo-1-p-tolyl-pentan-1-one (x) as a starting material. The yield was 82%. ¹H NMR δ 7.85 (d, 2H), 7.25 (d, 2H), 7.10 (dt, 1H), 6.88 (dt, 1H), 2.39 (s, 3H), 2.32 (m, 2H), 1.13 (t, 3H); ¹³C NMR δ 190.3, 150.6, 143.2, 135.3, 129.0, 128.5, 124.7, 25.7, 21.5, 12.2.

### Example 66

1-(3-Iodo-phenyl)-pentan-1-one. This compound was prepared according to General Procedure C and was purified by column chromatography (3% EtOAc/hexanes). The yield was 29%. ¹H NMR δ 8.28 (t, 1H), 7.90 (m, 2H), 7.21 (t, 3H), 2.93 (t, 2H), 1.71 (m, 2H), 1.40 (m, 2H), 0.96 (t, 3H); ¹³C NMR δ 199.1, 141.6, 138.8, 137.0, 130.3, 127.1, 94.4, 38.3, 26.2, 22.4, 13.9.

### Example 67

1-(4-Iodo-phenyl)-pentan-1-one. This compound was prepared in very low yield by following General Procedure C. Friedel Crafts acylation of iodobenzene employing the “Perrier Method” (J. Chem. Soc. P1 2493, 1973) gave a mixture of compounds. The crude compound could be distilled from this mixture (Bp 112° C., 0.1 mmHg) and further purified by recrystallization from EtOH. The yield was 11%. ¹H NMR δ 7.82 (d, 2H), 7.67 (d, 2H), 2.92 (t, 2H), 1.71 (m, 2H), 1.40 (m, 2H), 0.95 (t, 3H).

### Example 68

1-o-Tolyl-pentan-1-one. This compound was prepared following General Procedure C and was purified by distillation (Bp 58-60° C., 0.05 mmHg). The yield was 75%. ¹H NMR δ 7.62 (m, 1H), 7.36 (m, 1H), 7.26 (m, 2H), 2.89 (t, 2H), 2.48 (s, 3H), 1.68 (m, 2H), 1.39 (m, 2H), 0.94 (t, 3H).

### Example 69

1-m-Tolyl-pentan-1-one. This compound was prepared following General Procedure C and was purified by distillation (Bp 64-68° C., 0.1 mmHg). The yield was 98% ¹H NMR δ 7.86 (d, 2H), 7.26 (d, 2H), 2.94 (t, 2H), 2.41 (s, 3H), 1.71 (m, 2H), 1.41 (m, 2H), 0.95 (t, 3H).

### Example 70

Dopamine Transporter Occupancy of Pyrovalerone Analogs

Entry of compounds into brain is an important criterion for assessing the diagnostic and therapeutic potential of compounds targeted to the central nervous system. Access of compounds into brain targets may be attenuated by rapid peripheral metabolism, by sequestration by proteins or organs in peripheral tissues, or by the blood brain barrier. Brain imaging is an efficient method for determining the biological potential of a novel compound designed to affect brain function or to image the brain.

As the compounds of the invention are high affinity ligands for the dopamine transporter, we determined whether they occupy the dopamine transporter in living brain within 1 hour of administration. To monitor occupancy of the dopamine transporter, PET imaging was conducted with the high affinity dopamine transporter probe [11C]CFT ([11C]WIN 35,428). Rhesus monkeys were anesthetized with ketamine and xylazine and an indwelling intravenous catheter was placed in a leg vein. DAT density (binding potential) was acquired with [11C]CFT to obtain baseline levels. Immediately following completion of the imaging session, monkeys were administered the test compound intravenously via the indwelling catheter and PET imaging was conducted one hour after administration. Imaging data from the pre- and post-drug session were compared and occupancy was calculated on the basis of reduced [11C]CFT binding potential one hour or longer after administration of the compound. The following table (Table 1) summarizes pilot data from this study.

TABLE 1
Compound occupancy of the dopamine transporter,
as determined by PET imaging

| | DAT | | | [11C]CFT | |
|---|---|---|---|---|---|
| | Affinity | Monkey | [11C]CFT | with | % |
| Compound | (nM) | # | Baseline | Compound | Occupancy |
| O-2371 | 8 | 104-91 | 1.7481 | 0.4551 | 100* |
| | | | | | 74% |
| O-2387 | 13 | 533-99 | 2.1654 | 0.7885 | 64% |
| O-2390 | 8 | 307-97 | 1.3445 | 0.5898 | 56% |
| O-2419 | 8 | 540-99 | 2.3919 | 1.6025 | 33% |
| O-2442 | 3 | 183-96 | 2.1578 | 0.6112 | 100* |
| | | | | (cerebellum | |
| | | | | baseline) | 70% |

*Reduced to levels of cerebellum. If cerebellum levels are considered background, then compounds achieved full occupancy

As described in Table 1, the test compounds occupy the dopamine transporter in living brain, as detected by PET imaging. Compounds O-2371 and O-2442 were the most efficient in entering the brain and occupying the majority of DAT sites (using cerebellum as the negative control).

The present invention has been described in detail, including the preferred embodiments thereof. However, it will be appreciated that those skilled in the art, upon consideration of the present disclosure, may make modifications and/or improvements of this invention and still be within the scope and spirit of this invention as set forth in the following claims.

All references cited are incorporated herein in their entirety by reference.

1. A compound represented by any of the following formulae: 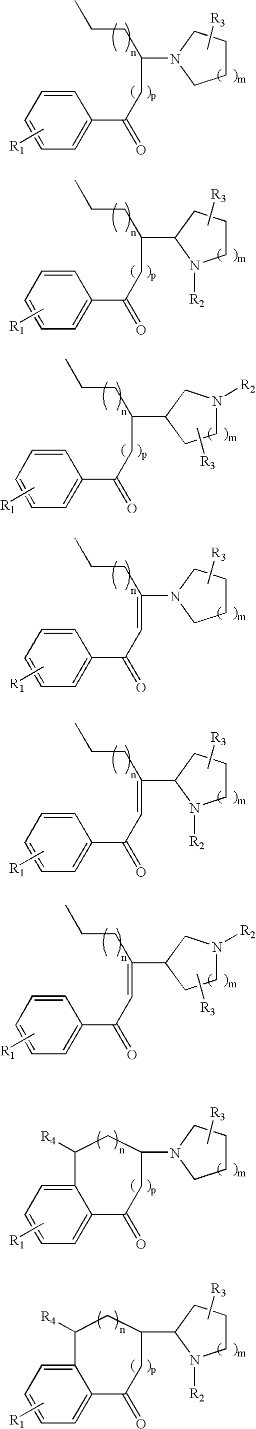 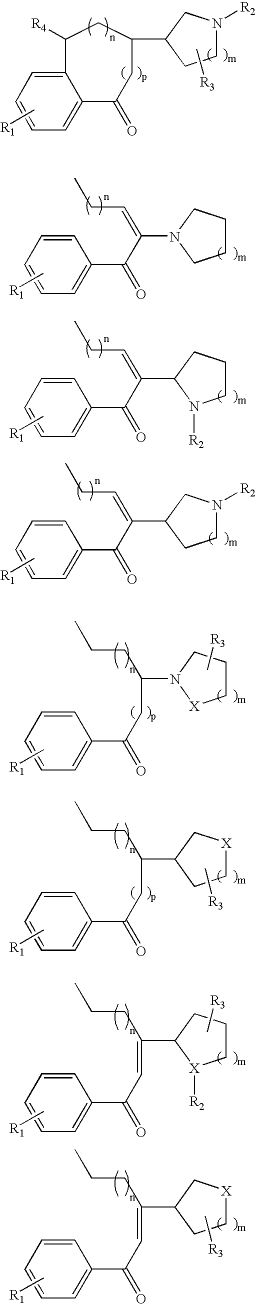 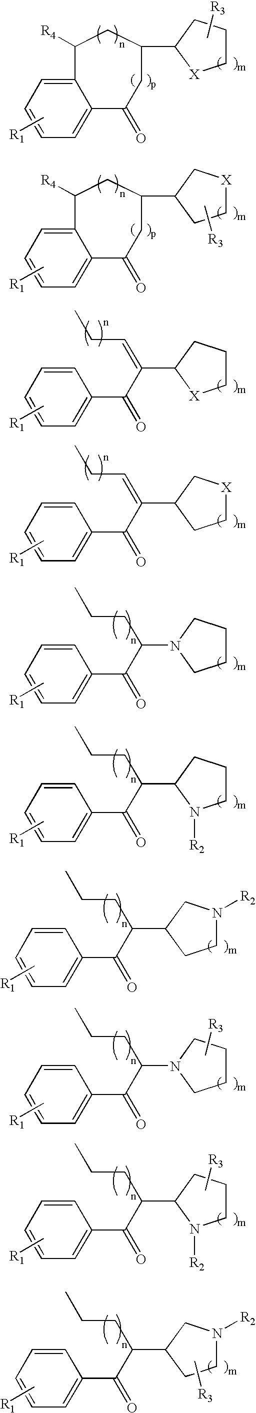 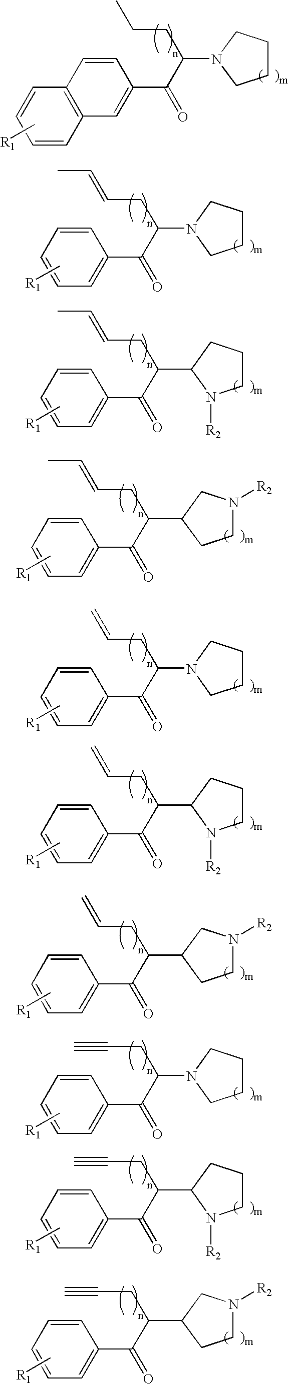 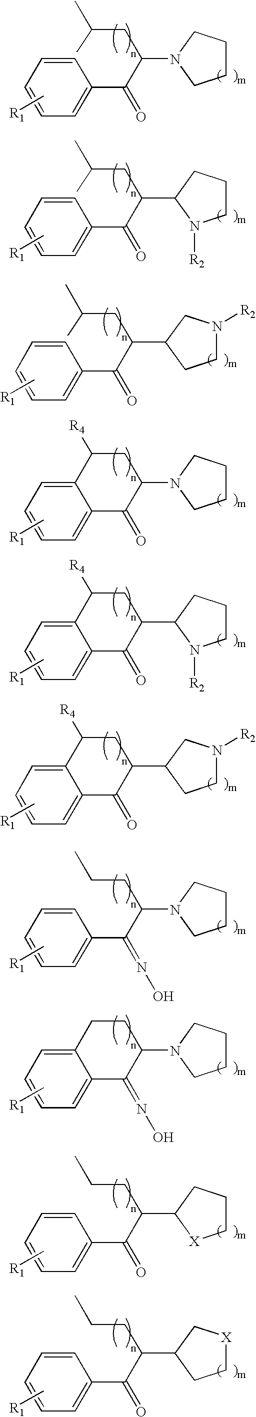 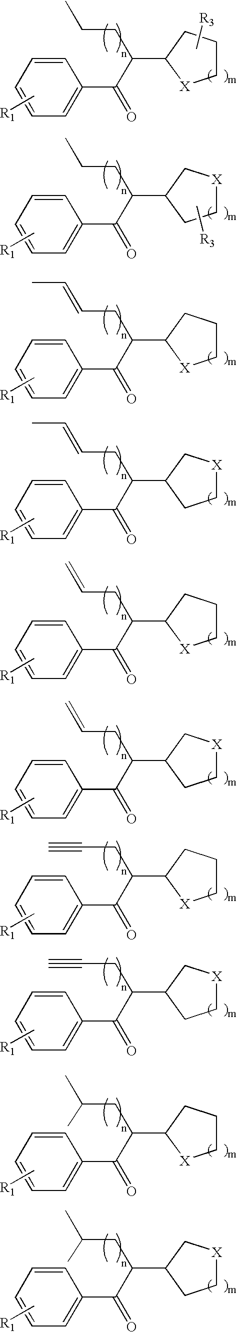 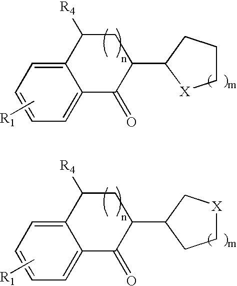
wherein,
R₁=one to four substituents independently selected from the group consisting of H, halogen, substituted or unsubstituted alkyl, substituted or unsubstituted alkoxy, substituted or unsubstituted alkenyl, substituted or unsubstituted alkenyloxy, substituted or unsubstituted alkynyl, substituted or unsubstituted alkynyloxy, (CH₂)ₙ—Ar, OH, OC(O)-alkyl; CF₃; NO₂; NH₂; CN; NHCOCH₃; CO-alkyl, CH₂OH, (CH₂)ₙOR₂ (in which n is 1 to 4) and (CH₂)ₙOCOR₂; (in which n is 1 to 4);
R₂═H, substituted or unsubstituted alkyl, substituted or unsubstituted alkoxy, substituted or unsubstituted alkenyl, substituted or unsubstituted alkenyloxy, substituted or unsubstituted alkynyl, substituted or unsubstituted alkynyloxy, or CH₂ArR₁;
R₃=one or two substituents independently selected from the group consisting of H, halogen, substituted or unsubstituted alkyl, substituted or unsubstituted alkoxy, substituted or unsubstituted alkenyl, substituted or unsubstituted alkenyloxy, substituted or unsubstituted alkynyl, substituted or unsubstituted alkynyloxy, OH, (CH₂)ₙArR₁; CF₃; NO₂; NH₂; CN; NHCOCH₃, CO-alkyl, CH₂OH, (CH₂)ₙOR₂ (in which n is 1 to 4) and (CH₂)ₙOCOR₂ (in which n is 1 to 4);
R₄═H, substituted or unsubstituted alkyl, substituted or unsubstituted alkoxy (preferably methoxy), substituted or unsubstituted alkenyl, substituted or unsubstituted alkenyloxy, substituted or unsubstituted alkynyl, substituted or unsubstituted alkynyloxy, OH, OC(O)-alkyl; CF₃; NO₂; NH₂; CN; NHCO-alkyl, COCH₃, CH₂OH, (CH₂)ₙOR₂ (in which n is 1 to 4) and (CH₂)ₙOCOR₂ (in which n is 1 to 4);
Ar is an aromatic group;
n=0-4;
m,p=0-2; and
X═O, CH₂, S, SO₂, or SO;
or a pharmaceutically acceptable salt thereof,
with the proviso that, when the compound is a racemic mixture, the compound is not α-pyrrolidino-valerophenone, pyrovalerone, 1-phenyl-2-pyrrolidino-3-methylbutan-1-one, 1-(p-methoxy-phenyl)-2-pyrrolidino-pentan-1-one, 1-(p-hydroxy-phenyl)-2-pyrrolidino-pentan-1-one, 1-phenyl-2-pyrrolidino-butan-1-one, 1-phenyl-2-pyrrolidino-heptan-1-one, -(p-chloro-phenyl)-2-pyrrolidino-pentan-1-one, 1-(m-methyl-phenyl)-2-pyrrolidino-pentan-1-one, 1-phenyl-2-pyrrolidino-nonan-1-one, 1-(p-methoxy-phenyl)-2-pyrrolidino-hexan-1-one, or α-(2′-methyl-pyrrolidino)-valerophenone.

2. A compound represented by the structure: 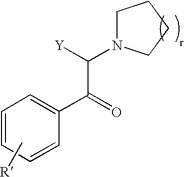
in which
R′ represents one to four substituents independently selected from the group consisting of H, halogen (preferably F, Br, Cl, or I), substituted or unsubstituted alkyl, substituted or unsubstituted alkoxy (preferably methoxy), substituted or unsubstituted alkenyl, substituted or unsubstituted alkenyloxy, substituted or unsubstituted alkynyl, substituted or unsubstituted alkynyloxy, (CH₂)ₙ—Ar, OH, OC(O)-alkyl, CF₃, NO₂, NH₂, CN, NHCOCH₃, CO-alkyl, CH₂OH, (CH₂)ₙOR₂ (in which n is 1 to 4) and (CH₂)ₙOCOR₂ (in which n is 1 to 4);
Y is an aliphatic group having from 1 to 8 carbons in a straight, branched, or cyclic aliphatic chain, and
r is 1 or 2; or a pharmaceutically acceptable salt thereof;
provided that: when the compound is a racemic mixture, 1) if Y is n-propyl, and r is 1, then R′ is not H, 4-methyl, 4-hydroxy, 4-methoxy, 4-chloro, or 3-methyl; and 2) if Y is ethyl, isopropyl, n-butyl, n-pentyl, or n-heptyl, and r is 1, then R′ is not H for every occurrence.

3. The compound of claim 2, in which the compound is the 2S-enantiomer.

4. The compound of claim 2, in which R′ is selected from the group consisting of 4-F, 4-Br, or 4-I.

5. The compound of claim 2, in which R′ represents 3,4-Cl, 3,4-OH, or 3,4-diacetoxy.

6. The compound of claim 2, in which R′ is 4-acetamido or R′ is 4-nitro.

7. The compound of claim 2, in which R′ is 2-methyl or 3-I.

8. The compound of claim 2, in which R′ is 4-hydroxymethyl or 4-C(O)O-alkyl

9. The compound of claim 2, in which R′ is 4-alkynyl.

10. The compound of claim 2, in which R′ is an aromatic ring attached at the 4-position.

11. The compound of claim 2, in which the compound is the 2-R enantiomer.

12. The compound of claim 3, in which R′ is 4-methyl.

13. The compound of claim 2, in which the aliphatic group is an allyl group.

14. The compound of claim 2, in which the aliphatic group is an ethyl group.

15. The compound of claim 2, in which the aliphatic group is an isobutyl group.

16. The compound of claim 2, in which the aliphatic group is an n-propyl group.

17-49. (canceled)

50. A compound represented by the structure: 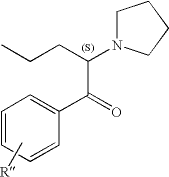
in which R″ represents one to four substituents selected from the group consisting of halogen, lower alkyl, lower alkenyl, lower alkynyl, aryl, hydroxy, —CF₃, nitro, amido, —(O)CO-alkyl and —C(O)O-alkyl;
and pharmaceutically acceptable salts thereof.

51. The compound of claim 50, in which R″ represents 4-alkyl.

52. A compound selected from the group consisting of
1-(4-Propynyl-phenyl)-2-pyrrolidin-1-yl-pentan-1-one
4-Methyl-2-pyrrolidin-1-yl-1 p-tolyl-pentan-1-one
1-(4-Iodo-phenyl)-2-pyrrolidin-1-yl-pentan-1-one
1-Naphthalen-2-yl-2-pyrrolidin-1-yl-pentan-1-one
2-Pyrrolidin-1-yl-1-m-tolyl-pentan-1-one
2-Pyrrolidin-1-yl-1-o-tolyl-pentan-1-one
2-Pyrrolidin-1-yl-1-(4-thiophen-2-yl-phenyl)-pentan-1-one
2-Pyrrolidin-1-yl-1-(4-furan-2-yl-phenyl)-pentan-1-one
2-Pyrrolidin-1-yl-1-(4-nitro-phenyl)-pentan-1-one
N-[4-(2-Pyrrolidin-1-yl-pentanoyl)-phenyl]-acetamide
2-Pyrrolidin-1-yl-1-(4′-bromo-phenyl)-pentan-1-one
2-Pyrrolidin-1-yl-1-(4′-hydroxy-phenyl)-pentan-1-one
2-Pyrrolidin-1-yl-1-(4′-methoxy-phenyl)-pentan-1-one
1-(3-Iodo-phenyl)-2-pyrrolidin-1-yl-pentan-1-one
2-Pyrrolidin-1-yl-1-(3,4-Dichloro-phenyl)-pentan-1-one
2-Pyrrolidin-1-yl-phenyl-pentan-1-one
2-Pyrrolidin-1-yl-1-(4′-fluoro-phenyl)-pentan-1-one
(S)-2-Pyrrolidin-1-yl-1-p-tolyl-pentan-1-one
1-(4-Hydroxymethyl-phenyl)-2-pyrrolidin-1-yl-pentan-1-one
4-(2-Pyrrolidin-1-yl-pentanoyl)-benzoic acid methyl ester and
1-(3,4-Dihydroxy-phenyl)-2-pyrrolidin-1-yl-pentan-1-one
and pharmaceutically acceptable salts thereof.

53-76. (canceled)
